SEQUENCE LISTING (18 sequences)

SEQ ID NO: 1 - DNA, 21 bp, Mus musculus
  source 1..21: mol_type = genomic DNA
agatcgctgtgaacggatttg

SEQ ID NO: 2 - DNA, 23 bp, Mus musculus
  source 1..23: mol_type = genomic DNA
tgtagaccatgtagttgaggtca

SEQ ID NO: 3 - DNA, 24 bp, Mus musculus
  source 1..24: mol_type = genomic DNA
gggccagtcagcatgatagaaaca

SEQ ID NO: 4 - DNA, 20 bp, Mus musculus
  source 1..20: mol_type = genomic DNA
acagcgcactgttcagagtt

SEQ ID NO: 5 - DNA, 20 bp, Herpes simplex virus
  source 1..20: mol_type = genomic DNA
agaaaatgcccacgctactg

SEQ ID NO: 6 - DNA, 20 bp, Herpes simplex virus
  source 1..20: mol_type = genomic DNA
attggcaagcagcccgtaaa

SEQ ID NO: 7 - DNA, 22 bp, Mus musculus
  source 1..22: mol_type = genomic DNA
aggatgcgtccaccaagaagct

SEQ ID NO: 8 - DNA, 22 bp, Mus musculus
  source 1..22: mol_type = genomic DNA
tccgtgtccacgtcagcaatca

SEQ ID NO: 9 - DNA, 20 bp, Mus musculus
  source 1..20: mol_type = genomic DNA
atgcacactctgcgatgaag

SEQ ID NO: 10 - DNA, 20 bp, Mus musculus
  source 1..20: mol_type = genomic DNA
cagtgttcacagccaggaga

SEQ ID NO: 11 - DNA, 23 bp, Mus musculus
  source 1..23: mol_type = genomic DNA
gaactgtgaggagatgagaaggc

SEQ ID NO: 12 - DNA, 22 bp, Mus musculus
  source 1..22: mol_type = genomic DNA
tggcttcgcttacaacgtgtgc

SEQ ID NO: 13 - DNA, 23 bp, Mus musculus
  source 1..23: mol_type = genomic DNA
ggagttctgactggaaagccgaa

SEQ ID NO: 14 - DNA, 22 bp, Mus musculus
  source 1..22: mol_type = genomic DNA
cttctggcaagccatctcctca

SEQ ID NO: 15 - DNA, 22 bp, Mus musculus
  source 1..22: mol_type = genomic DNA
gccacctatctgaatgaccacc

SEQ ID NO: 16 - DNA, 21 bp, Mus musculus
  source 1..21: mol_type = genomic DNA
aggaaccagcggttgaacggc

SEQ ID NO: 17 - DNA, 22 bp, Mus musculus
  source 1..22: mol_type = genomic DNA
gaggagaaacgccaaccaagag

SEQ ID NO: 18 - DNA, 22 bp, Mus musculus
  source 1..22: mol_type = genomic DNA
tttgtcctcggtggcgttatcc

(19) United States
(12) Patent Application Publication
(10) Pub. No.: US 2026/0234573 A1
(43) Pub. Date: Aug. 13, 2026

(54) COMPOSITION INCLUDING VIRUS LIKE PARTICLES (VLPS), A KIT, AND METHOD FOR ANTITUMORAL THERAPY

(71) Applicant: CNPEM - CENTRO NACIONAL DE PESQUISA EM ENERGIA E MATERIAIS, Campinas (BR)

(72) Inventors: Marcio Chaim Bajgelman, Campinas (BR); Isadora Ferraz Semionatto, Campinas (BR)

(21) Appl. No.: 19/409,210

(22) Filed: Dec. 4, 2025

Related U.S. Application Data
(60) Provisional application No. 63/727,778, filed on Dec. 4, 2024.

(51) Int. Cl. C12N 7/04; A61K 31/522; A61K 35/763; A61K 35/766; A61K 38/16; A61K 38/19

(52) U.S. Cl. CPC C12N 7/045; A61K 31/522; A61K 35/763; A61K 35/766; A61K 38/162; A61K 38/191; C12N 2740/15023; C12N 2740/15033

(57) ABSTRACT

A composition for use in antitumoral therapy, where the composition combines two virus like particles (VLPs) capable of promoting an abscopal effect. The composition can include: a VLP-derived from a viral capsid decorated with VSV-g envelope glycoprotein and loaded with Thymidine Kinase (HSV-TK) from viral herpes; a VLP-derived from a viral capsid decorated with CD40L; and/or a combination thereof. A kit is disclosed for antitumoral therapy, where the kit includes: a VLP-derived from a viral capsid decorated with VSV-g envelope glycoprotein and loaded with HSV-TK from viral herpes; a VLP-derived from viral capsid decorated with CD40L; and (c) an antiviral drug, where the VLPs are derived from a lentivirus capsid. A method is disclosed for antitumoral therapy, where the method involves administration of two VLPs capable of promoting an abscopal effect, and an antiviral drug.

### REFERENCE TO AN ELECTRONIC SEQUENCE LISTING

The present application contains a Sequence Listing XML submitted electronically in XML format via Patent Center. The Sequence Listing XML, created on Mar. 27, 2026, is named “20260327-SequenceListing.xml” and is 16,958 bytes in size. The Sequence Listing XML is part of the specification.

### FIELD

The present disclosure relates to a combined VLP strategy to antitumoral therapy, capable of promoting an abscopal effect.

### BACKGROUND OF THE DISCLOSURE

Nanomedicine has gained great prominence in the prevention, diagnosis and treatment of numerous diseases such as cancer, autoimmune diseases, hepatitis, hypercholesterolemia and many other conditions (SCHMID et al., 2018, PARK et al., 2016; KIM et al., 2002; PARK et al., 2016; KIM et al., 2002, HOU et al., 2020). The use of nanoparticles as a drug and biopharmaceutical delivery system is proposed as a promising therapeutic approach for the targeted and controlled delivery of medicines to specific locations in the body (PATRA et al., 2018).

Currently, there are several types of nanosystems used in the oriented and personalized delivery of biomolecules to the site of action. Liposomes, polymers and inorganic particles have been used to overcome the limitations of conventional drug administration methods: non-specific biodistribution, poor bioavailability and important side effects (HONG et al., 2020; BONVALOT et al., 2019; LUO et al; 2017; SULTANA et al., 2022). These nanoparticles, for the most part, are biocompatible, stable, biodegradable, water-soluble, simple to formulate, easily engineered and have high bioavailability (MITCHELL et al., 2021). However, the implementation of these classes of nanoparticles to deliver therapeutic substances still faces some challenges in the clinic, such as a low degree of encapsulation and early release of the drug, reducing its biodistribution (TOMEH et al., 2021; LI; LI; ZHAO, 2020). In view of this, the development of new nanocarriers for the targeted delivery of medicines is necessary.

Viral nanoparticles are potential candidates as biological platforms for delivering biomolecules (CHUNG; CAI; STEINMETZ, 2020 GEORGE et al., 2017).

They can be engineered to evade the host immune system, they can be internalized by target cells; have the ability to escape from endosomal vesicles before their degradation; and are capable of releasing the active substance into the cytoplasm or introducing the gene of interest into the cell genome (FEDOTOV et al., 2022; CHAN et al., 2021; GHOSH; BANERJEE, 2021). The Herpes simplex virus and several viral vectors—retroviral, adenoviral, adeno-associated and lentiviral, have been extensively studied as gene transfer and vaccination tools (BULCHA et al., 2021).

Currently, at least four drugs based on viral vectors have gained wide application for gene therapy and have been regularly approved for use in the United States by the respective regulatory body FDA. Glybera (UniQure), Imyligic (Biovex Inc.), Luxturna (Spark Therapeutics, Inc.), Zolgensma (Novartis) are examples of viral vector-based therapies for the treatment of diseases such as chronic pancreatitis due to congenital lipase deficiency, melanoma metastatic, retinal degeneration and pediatric spinal muscular atrophy, respectively (HAMPSON et al., 2018; POL; KROEMER; GALLUZZI, 2016; MAGUIRE et al., 2021; MAGUIRE et al., 2021; MERCURI et al., 2021). The Brazilian Food and Drug Agency (ANVISA) had already approved the tests of Luxturna and Zolgensma. As prophylactic agents, adenoviral vectors became famous during the COVID-19 pandemic (KNOLL; WONODI, 2021).

Although the use of viral vectors has considerable advantages, the mutagenic and carcinogenic potential and the long-term expression of the transgene are some of the main obstacles to implementing this delivery system in the medical clinic (HACEIN-BEY-ABINA et al., 2002; BRUNETTI-PIERRI et al., 2013). Therefore, the use of virus-like particles that mimic the general structure of the virus, but do not contain infectious genetic material, can function as a safer alternative for delivering macromolecules and vaccination (NOORAEI et al., 2021; BAYER; BLUMBERG; WERNER, 1968).

VLPs (Virus Like Particles) are nanometric structures, measuring between 20 and 200 ηm, composed of structural viral proteins that reproduce the organization and conformation of the native virus, but lack the infectious viral genetic material (MANOLOVA et al., 2008). These particles have an inherent property for 19 self-assembly, in addition to being non-replicating and non-infectious (TARIQ et al., 2022; SWANN et al., 2020).

VLPs (Virus Like Particles) are nanometric structures, measuring between 20 and 200 ηm, composed of structural viral proteins that reproduce the organization and conformation of the native virus, but lack the infectious viral genetic material (MANOLOVA et al., 2008). These particles have an inherent property for self-assembly, in addition to being non-replicating and non-infectious (TARIQ et al., 2022; SWANN et al., 2020).

VLPs have become important tools in the biomedical field. Due to their immunogenic and antigenic properties, VLPs are widely used in the production of vaccines for different diseases, such as Hepatitis B (HBV) (Engerix-B®; GenHevac B®, Recombivax®), HPV (Gardasil® and Cervarix®) and influenza A (Inflexal®), already licensed by the FDA and commercialized (ELHANAN et al., 2018; MCLEMORE, 2006; PITOISET et al., 2017; SZAREWSKI, 2012b; YETGIN et al., 2007; ZHAO et al., 2011a).

VLPs can stimulate strong humoral and cellular responses (YILMAZ et al., 2022). These particles are highly efficient in activating CD4+ T and CD8+ T cells even in the absence of infection or intracellular replication (BRAUN et al., 2012; HEMANN; KANG; LEGGE, 2013). Because they may display a high number of repetitive antigen sequences on their surface, in addition to their nanometric size, VLPs may be easily recognized by antigen-presenting cells. This repetitive array of antigens resembles pathogen recognition patterns (PAMP), leading to the potent activation of dendritic cells (DCs), which begin to secrete cytokines and express high levels of co-stimulatory molecules (KANEKIYO; BUCK, 2017). These cells internalize the VLPs, process and transport the internalized proteins to the lymph nodes and present the antigenic peptides to CD4+ T lymphocytes via MHC II and to CD8+ T lymphocytes via cross-presentation through the MHC class I molecule (GOMES et al., 2022; SERRADELL et al., 2019).

In addition to the ability to induce cellular responses mediated by CD4+ T cells and cytotoxic T cells, VLPs may be effective in inducing B cell responses (JEGERLEHNER et al., 2002). B lymphocytes recognize antigen-dense sequences arranged on the surface of VLPs, leading to the proliferation and migration of B cells, production and secretion of IgM and IgG antibodies, and generation of long-lived memory cells (BESSA et al., 2012).

Although VLPs have intrinsic characteristics that facilitate the induction of immune responses without the need for adjuvants, the introduction of antigenic peptides or immunomodulatory molecules into VLPs can increase their immunogenicity and provoke strong specific immune responses (CIMICA; GALARZA, 2017).

VLPs have emerged as a potentially promising platform for delivering therapeutic agents (BRENDLE et al., 2021). They can act as a powerful resource for transporting different molecules such as drugs, genes, diagnostic agents, proteins and peptides to different regions of the organism (BIABANIKHANKAHDANI et al., 2016; BANSKOTA et al., 2022; LEONG et al., 2010).

Similar to native viruses, VLPs are target specific, efficiently penetrate the host cell, are biocompatible and biodegradable (TARIQ et al., 2022; SHAN et al., 2018). The receptors exposed on the surface of VLPs facilitate the internalization of these particles by the target cell, reducing their side effects and toxicity (KUMAR et al., 2022; ZHAO et al., 2011a; THONG et al., 2019).

VLPs have the ability to anchor or encapsulate different proteins, nucleic acids and other small molecules (BANSKOTA et al., 2022a; ZILBERZWIGE-TAL et al., 2021). Upon reaching specific cells, tissues or organs, these nanoparticles interact with receptors present in the cell plasma membrane and can be endocytosed into the host cell, traveling through the canonical endocytic pathway (primary endosomes, late endosomes and endolysosomes (MERCER; SCHELHAAS; HELENIUS, 2010). However, as the endosomal vesicles mature, the endosomal pH decreases, leading to conformational changes in the protein structure of the VLPs that, consequently, become able to escape lysosomal degradation (CASTRO et al., 2021). This characteristic is unique to viruses and VLPs, making them innate delivery systems.

Chemical or genetic modifications of proteins contained on the surface or inside VLPs have become a very efficient strategy to improve the therapeutic efficacy, bioavailability and interaction of these particles with specific cells (TAGIT et al., 2017; CHEN et al., 2019a). Many changes in the protein repertoire of VLPs are intended to reduce their immunogenic potential and increase their targeting to the target site (NETTER et al., 2003; GRATAITONG et al., 2021). The intrinsic immunogenicity of VLPs can be mitigated with the insertion of polyethylene glycol (PEG) molecules at their end, which facilitates evasion of the immune system and increases the efficiency of therapeutic delivery (BARENHOLZ, 2012; VERONESE; MERO, 2008). Another device that has been investigated to reduce activation of the immune system and the removal of circulating VLPs is the construction of chimeric particles that express the CD47 glycoprotein, which is present in all mammalian cells and is used by leukocytes to distinguish self from non-self. (SCHWARZ et al., 2015). In addition to lower immunogenicity, VLPs can be directed by the expression of specific ligands that confer tropism to target cells. A recent study published by the inventor's research group demonstrated that multivalent VLPs constructed with costimulatory proteins and a peptide that offers tropism to prostate cancer cells were efficient in suppressing tumor growth and increasing the infiltration of CD8+ T lymphocytes in the tumor microenvironment (PALAMETA et al., 2022). Just like peptides, VLPs can be conjugated to antibodies, aptamers and other surface proteins that improve targeting specificity and delivery efficiency (LEIJA-MONTOYA et al., 2014; KIM et al., 2019; HAMILTON et al., 2021; KATO et al., 2015).

Pseudotyping, which consists of the use of heterologous envelopes, is another method adopted to modify the tropism of VLPs and amplify their delivery potential (BURNS et al., 1993). Through this technique, native proteins present on the surface of VLPs are replaced by a viral envelope of interest, which restricts or expands the selectivity of these particles (ROBERT et al., 2017). A protein frequently used for pseudotyping of retroviral vectors, for example, is vesicular stomatitis virus (VSV)-derived G glycoprotein, which mediates entry into the target cell through LDL receptors (KIM et al., 2017). VSV-G acquired a pantotropic profile and high host cell transduction capacity (GUTIERREZ-GUERRERO; COSSET; VERHOEYEN, 2020).

The immune system plays a crucial role in suppressing tumor development and growth (GONZALEZ; HAGERLING; WERB, 2018). Immune system cells are capable of detecting and eliminating cells transformed as a result of multiple mutations and structural modifications of the cell genome (TOMASETTI; LI; VOGELSTEIN, 2017). In the cancer cell, this accumulation of genetic changes leads to the appearance of new tumor-specific proteins or induces the overexpression of endogenous proteins (BEERENWINKEL et al., 2007). These proteins, also known as neoantigens (TSA) or tumor-associated antigens (TAA), can effectively activate and stimulate T cells, inducing a potent antitumor immune response (YU et al., 2004).

In the early stages of tumorigenesis, T lymphocytes, through their T cell receptors (TCRs), recognize tumor antigens complexed with MHC class I and II molecules expressed on antigen presenting cells (APCs) (HUANG et al., 1994). The interaction between T cells and APCs promotes a cellular immune response that controls tumor development and progression (PENTCHEVA-HOANG et al., 2004). APCs, which include dendritic cells (DC), macrophages and B lymphocytes, are necessary for the generation of an adaptive immune response, bridging the gap between innate and acquired immunity (BANCHEREAU; STEINMAN, 1998; PAGLIA et al., 1996). These cells participate in both antigen processing and presentation, emitting co-stimulatory signals to T cells (PAGLIA et al., 1996; SCHIRRMACHER et al., 2015).

Costimulatory signals are necessary for the complete activation of T cells (CHEN; FLIES, 2013b). In addition to the first activation signal, provided by the interaction between TCRs and the peptide-MHC complex, a second, antigen-independent signal is essential for T cells to proliferate, differentiate, mediate effector functions and acquire memory (BUCHAN et al., 2015). The second activation signal is provided by a wide range of costimulatory molecules on the surface of APCs, which interact with specific costimulatory receptors expressed on T lymphocytes (BRETSCHER, 1999; CHEN; FLIES, 2013a). When both signals are orchestrated synchronously, together with the action of pro-inflammatory cytokines (third signal), T cells become completely activated and provoke an effective immune response against the tumor (DRIESSENS; KLINE; GAJEWSKI, 2009; CURTSINGER; MESCHER, 2010).

The CD28-B7-1/B7-2 (CD80/CD86) signaling pathway is the best characterized T cell costimulatory pathway, being crucial for the initiation of adaptive immune responses (PENTCHEVA-HOANG et al., 2004). CD28 is constitutively expressed on T cells and its interaction with the CD80/CD86 ligands present on APCs induces the secretion of cytokines, clonal expansion and inhibition of apoptosis (SAVOLDO et al., 2011; ESENSTEN et al., 2016; PASTOR et al., 2013). Consequent to CD28-B7-1/B7-2 (CD80/CD86) signaling, other pairs of costimulatory molecules are upregulated in T lymphocytes and APCs. In particular, members of the TNF family of proteins, including the receptors 4-1 BB (CD137), OX40 (CD134), CD40 and CD27 and their ligands 4-1 BB ligand, OX40 ligand, CD40 ligand and CD70, respectively, have been shown to extreme importance for the amplification of immune responses mediated by T cells (MASCARELLI et al., 2021; OBA et al., 2020; BUCHAN et al., 2015; ZHANG et al., 2007).

However, at more advanced stages of tumor development, the most aggressive neoplastic cells, naturally selected by their tumor microenvironment, begin to evade immunological attacks and proliferate uncontrollably (DUPAGE et al., 2012). At this stage, T cells lack appropriate co-stimulatory signals, becoming unresponsive and immunologically tolerant to the presence of neoplastic cells (LOOS et al., 2009).

In this scenario, immunotherapies emerge as an effective therapeutic approach to overcome tumor immunotolerance and enhance immune responses against the tumor (COLEY, 1898). Currently, the most commonly used immunotherapeutic strategies aim to provide co-stimulatory signals or block inhibitory signals in effector T cells. Immune checkpoint inhibitors (ICIs), antitumor vaccines, adoptive T cell transfer, cytokine therapies and oncolytic virus therapies are the main types of immunotherapies applied for the treatment of cancer (JEONG; PARK, 2020).

Antitumor vaccines encompass several resources to boost the immune system to destroy neoplastic cells (PODAZA et al., 2020). The production of therapeutic vaccines based on neoantigens for T cell activation has been extensively explored, and several studies are in the clinical phase (NCT03639714, NCT03223103 and NCT02721043) (PALMER et al., 2022; WANG et al., 2021; BLAZQUEZ et al., 2019). Another example of an antitumor vaccine is the pulse of dendritic cells with tumor antigens (GROSS et al., 2017). These cells process tumor-associated antigens (TAAs) and present their epitopes to T cells, leading to a potent cytotoxic T lymphocyte response (GROSS et al., 2017). Sipuleucel-T is a representative and pioneering example in cancer immunotherapy, of dendritic cell vaccines approved for the treatment of prostate cancer (KANTOFF et al., 2010). Furthermore, tumor cells themselves engineered with immunomodulators can also be used as vaccines. In previous work, the inventor's research group also demonstrated the efficiency of antitumor vaccines derived from genetically modified cancer cells with the molecules 4-1 BBL, OX40L and GM-CSF, in inhibiting tumor growth and even generating a long-term response after a second challenge, suggesting the generation of an immunological memory associated with specific combinations of immunomodulators (MANRIQUE-RINCON et al., 2017).

Immunotherapy has also taken advantage of the infectious potential of viruses to destroy cancer (HOWELLS et al., 2017). Oncolytic virus therapy uses viruses that selectively attack tumor cells, sparing normal cells (JIANG et al., 2017). Generally, this immunotherapy is combined with other immunotherapy treatments with the aim of enhancing antitumor immune responses (VANSEGGELEN et al., 2015). Talimogene laherparepvec (T-VEC), already approved for clinical use by the FDA in 2015, is an example of an oncolytic virus-based therapy for the local treatment of advanced-stage melanoma (LOUIE et al., 2020). This oncolytic virus is composed of the Herpes simplex virus type 1 genetically modified to lyse specific tumor cells and secrete the cytokine GM-CSF, amplifying antitumor immunity (LOUIE et al., 2019). The use of recombinant cytokines in conjunction with other immunotherapies can generate potent and coordinated immune responses against the target antigen (AMATO, 1999; KIRKWOOD et al., 2000). The administration of IL-2 and IFN-α has proven efficient in stimulating the immune system and causing regression of several types of cancer (ORECCHIA et al., 2015; AMATO, 1999; KIRKWOOD et al., 2000).

VLPs can be explored as a possible tool for cancer immunotherapy (BOLLI et al., 2018). Its innate biocompatibility, moderate immunogenicity, biodegradability, external and internal chemical modification capacity, and safety offer cancer treatment a highly customizable tool for the delivery of multiple immunotherapeutic components (CHUNG; CAI; STEINMETZ, 2020b).

Modifying the external surface of VLPs with immunomodulators from the tumor necrosis factor superfamily (TNFSF) can be a direct and effective method of manipulating the immune system to attack tumor cells (PALAMETA et al., 2022). It has previously been demonstrated by the inventors that VLPs engineered with the co-stimulators 4-1 BBL and OX40L and the membrane-anchored cytokine GM-CSF stimulate the proliferation of effector T lymphocytes, inhibit the suppressive function of regulatory T cells (T reg) and potentiate the elimination of cancer cells (PALAMETA et al., 2022). The delivery of the CD40L protein to the membrane of VLPs may function as an efficient way to induce strong Th1 immune responses, by stimulating innate and acquired immunity against cancer (SZEKERES et al., 2011; FILBERT et al., 2021; GOODALL et al., 2021).

The CD40/CD40L signaling pathway is essential for T cell activation (FERRIS et al., 2020). The CD40 receptor, also known as TNFRF5, is a 48 kDa type I transmembrane protein, expressed in several types of cells: dendritic cells (DCs), macrophages, B lymphocytes, platelets, endothelial cells, fibroblasts, epithelial cells and in different types of tumor cells, such as breast, melanoma, ovary, pancreas, colon and lung (INWALD et al., 2003; FLAXENBURG et al., 2004; ATAMAS et al., 2002; LEE et al., 2003; BATES et al., 2023) The CD40 receptor is expressed as monomers that trimerize after interacting with its ligand (TANG et al., 2021; WERNEBURG et al., 2001). The CD40 ligand, known as CD40L or CD154, is a 33 kDa type II transmembrane molecule, found in the form of trimers, mainly on activated CD4+ T cells, and, to a lesser extent, on activated B and NK cells (JYOTHI; KHAR, 2000; POSSAMAI et al., 2021; TAY et al., 2017).

Signaling between CD40 and CD40L is bidirectional. Both APCs and T cells benefit from CD40/CD40L costimulatory signals (MA; CLARK, 2009). On inactivated (immature) DCs, CD40 is constitutively expressed at lower levels; however, in activated (mature) DCs the expression of the receptor is increased, therefore serving as a marker of dendritic cell activation (XING et al., 2011). Thus, the encounter of DCs with microbial products, pathogens and apoptotic cells results in greater expression of CD40 (OBA et al., 2020). By associating with CD40L, CD40/CD40L signaling in DCs increases antigen presentation, promotes the production of pro-inflammatory cytokines (IL-12, TNF, IL-6 and IL1), the induction of costimulatory molecules (MHC II, CD80, CD86, CD83, 4-1 BBL and OX40L) and chemokine receptors that control migration to lymph nodes, such as CCR7, and facilitates the cross-presentation of antigens to CD8+ T cells, polarizing the response of T lymphocytes towards a Th1 profile (PAGLIA et al., 1996; TAY et al., 2017. In T cells, CD40L expression is basically restricted to activated CD4+ T cells, although a small population of CD8+ T lymphocytes express the ligand. CD40L signals contribute to the activation and effector action of T cells, which proliferate and secrete large amounts of cytokines, such as IFN-γ and TNF-α (BLAIR et al., 2000; QUEZADA et al., 2004). Furthermore, increased CD40L mRNA synthesis in CD4+ T cells is inversely correlated with the expression of FoxP3, a transcription factor essential for development of regulatory T cells (LOZANO et al., 2015). Another important function mediated by CD40L through CD4+ T lymphocytes is the licensing of dendritic cells in the activation of CD8+ T cells to become potent Cytotoxic T Lymphocytes (CTLs) (MORRISON et al., 2020a). CD40/CD40L signaling is essential for CTLs to promote a strong antitumor response and generate immunological memory against cancer (STUMBLES et al., 2004; MARIGO et al., 2016; BOURGEOIS; ROCHA; TANCHOT, 2002). Studies with CAR-T cells engineered to constitutively express CD40L showed greater efficiency in licensing DCs, ability to recruit CD8+ T lymphocytes to the tumor site and antitumor efficacy (KUHN et al., 2019).

CD40 signals are also important for the tumoricidal function of macrophages (LUM et al., 2006). The activation of macrophages through the CD40/CD40L interaction induces these cells to produce nitric oxide (NO) and proinflammatory cytokines, such as TNF-α and INF-α, which lead to tumor cell death (CHEN et al., 2019b; LUM et al., 2006). It is well established that the presence of anti-CD40 agonist antibodies in the immunosuppressive tumor microenvironment can polarize tumor-associated macrophages (TAM), characterized by an M2 profile that promotes tumor progression, to an M1 response profile that mediates the destruction of cancer cells as part of the antitumor innate immune response (YANG et al., 2020; OSHI et al., 2020; JENSEN et al., 2015). In addition to the immunomodulatory function of CD40, CD40/CD40L signaling can induce apoptosis of neoplastic cells. The ligation of CD40 on tumor cells increases the expression of pro-apoptotic genes such as Fas and Bax, and induces the activation of caspase 3, which coordinates the destruction of cellular structures (ELIOPOULOS et al., 2000a; FENG; WANG, 2020). In contrast, Bcl-X and Ki67, which are genes overexpressed in tumor cells with a high proliferation rate, have their expression reduced in response to the effects of CD40 (CHONAN et al., 2015; FENG; WANG, 2020).

In addition to carrying immunomodulatory molecules on their surface, VLPs could also carry out the intracellular delivery of nucleic acids or proteins that lead to the death of tumor cells (HAMILTON et al., 2021; CHOU et al., 2010). The transport of suicide genes into the cytoplasm of tumor cells through VLPs could induce cancer cell death and amplify antitumor immune responses.

Suicide gene therapy is based on the introduction of genes derived from certain viruses or bacteria into tumor cells, so that they are exclusively capable of encoding proteins that convert non-toxic compounds (prodrugs) into lethal drugs. for their survival (WANG; CANINE; HATEFI, 2011). The thymidine kinase gene of Herpes simplex virus type I (HSV-TK) is a widely used suicide gene (IWASAWA et al., 2019). This gene, not expressed in mammalian cells, is capable of converting the prodrug ganciclovir (GCV) into a toxic metabolite, which functions as an acyclic and synthetic analogue of the precursor 2′-deoxy-guanosine, necessary for DNA synthesis (YI et al., 2018a). The incorporation of this cytotoxic metabolite into the nascent DNA chain prevents the replication of the molecule, leading to cell apoptosis (YI et al., 2018b). Furthermore, the action of the HSV-TK/GCV system is amplified by the bystander effect that allows the toxic compound to diffuse to other tumor cells through intercellular GAP junctions (OISHI et al., 2022; ZHANG et al., 2014 et al., 2014; LI et al., 2011). In this way, not only tumor cells that express the HSV-TK enzyme, but also those that have not been transduced with the viral thymidine kinase gene, are killed (LI et al., 2011).

It is interesting to note that in addition to the local effect, the HSV-TK/GCV system can also mediate abscopal effects, systemically promoting the activation of the immune response against the tumor (WILSON et al., 1996; PIERREFITE-CARLE, 2002); When cancer cells are killed by the HSV-TK/GCV system, they can release TAAs that function as damage-associated molecular patterns (DAMP) (ANDTBACKA et al., 2015); In response to the recognition of DAMPs by their receptors, dendritic cells begin to secrete pro-inflammatory cytokines and increase the expression of costimulatory proteins, which promote the activation and proliferation of T cells capable of destroying the tumor (TEH et al., 2015).

### BRIEF DESCRIPTION OF DRAWINGS

Various aspects and features of the disclosure are described herein with reference to the drawings.

FIG. 1 shows the death of tumor cells engineered with the viral enzyme thymidine kinase by the HSV-TK/GCV system, according to an example embodiment of the present disclosure.

FIG. 2 shows the HSV-TK/GCV system inhibiting the proliferation of B16F10-TK cells, according to an example embodiment of the present disclosure.

FIG. 3 shows a co-culture assay to evaluate the bystander effect of the HSV-TK/GCV system on cancer cells, according to an example embodiment of the present disclosure.

FIG. 4 shows that the tumor cells killed by the HSV-TK/GCV system release cellular debris and tumor antigens into the culture medium, according to an example embodiment of the present disclosure.

FIG. 5 shows the bone marrow-derived dendritic cells activate in the presence of fragments of tumor cells killed by the HSV-TK/GCV system, according to an example embodiment of the present disclosure.

FIG. 6 shows the measurement of the concentration and average size of VLPs VSVG-TK and CD40L preparations using the NTA equipment, according to an example embodiment of the present disclosure.

FIG. 7 shows a transmission electron cryomicroscopy (Cryo-EM) for morphological analysis of the VLP VSVG-TK and VLP CD40L preparation, according to an example embodiment of the present disclosure.

FIG. 8 shows the characterization of CD40L VLPs, according to an example embodiment of the present disclosure.

FIG. 9 shows that the VSVG-TK VLPs carry the HSV-TK suicide enzyme within them, according to an example embodiment of the present disclosure.

FIG. 10 shows a transduction test with lentiviral preparations encoding GFP, produced with different envelopes, according to an example embodiment of the present disclosure.

FIG. 11 shows that the VSVG-TK VLPs drive the death of murine melanoma cells by the HSV-TK/GCV system, according to an example embodiment of the present disclosure.

FIG. 12 shows that the VSVG-TK VLPs drive the death of murine triple-negative breast cancer cells by the HSV-TK/GCV system, according to an example embodiment of the present disclosure.

FIGS. 13A and 13B show that the VSVG-TK VLPs in association with the prodrug ganciclovir (GCV) promote a decrease in the viability of tumor cells, according to an example embodiment of the present disclosure.

FIG. 14 shows that the VSVG-TK VLPs lead to tumor cell apoptosis by the HSV-TK/GCV system, according to an example embodiment of the present disclosure.

FIG. 15 shows that the supernatant from dead tumor cells after treatment with VSVG-TK VLPs and ganciclovir releases cellular fragments that activate BMDCs, according to an example embodiment of the present disclosure.

FIG. 16 shows that the CD40L-engineered VLPs induce activation of BMDCs, according to an example embodiment of the present disclosure.

FIG. 17 shows that the CD40L VLPs have tropism for tumor cells, according to an example embodiment of the present disclosure.

FIG. 18 shows that the CD40L VLPs induce apoptosis in B16F10 tumor cells, according to an example embodiment of the present disclosure.

FIG. 19 shows that the CD40L VLPs induce apoptosis in 4T1 tumor cells, according to an example embodiment of the present disclosure.

FIG. 20 shows that the CD40L VLPs modulate the expression of genes that induce apoptosis and inhibit the expression of genes that stimulate cell proliferation, according to an example embodiment of the present disclosure.

FIGS. 21A and 21B show that the treatment with VSVG-TK and CD40L VLPs decreases tumor growth, according to an example embodiment of the present disclosure.

FIG. 22 shows that the Local administration of VSVG-TK VLPs induced an increase in the intratumoral amount of CD4 and CD8, according to an example embodiment of the present disclosure.

FIG. 23 shows that the spleen of animals treated with the combination of VSVG-TK VLPs and CD40L VLPs had an increased number of CD4+T and CD8+ T lymphocytes, according to an example embodiment of the present disclosure.

### DETAILED DESCRIPTION

Within this context, the present disclosure provides a composition comprising the combination of two virus like particles (VLPs), capable of promoting an abscopal effect.

Such VLPs are preferably selected from:
VLP VSVg-TK—particle derived from viral capsid, decorated with VSV-g envelope glycoprotein and loaded with Thymidine Kinase (HSV-TK) from viral herpes. The VLP is injected into the tumor site, and due to the VSV-G envelope, which has a broad spectrum of recognition for various cell types. It binds to the tumor cell and it is internalized by it. The HSV-TK protein, which is carried by the VLP, is then released into the cell. HSV-TK without Ganciclovir does not present cytotoxicity, but in the presence of Ganciclovir it converts the molecule into a cytotoxic agent that kills the tumor cell. The HSV-TK/Ganciclovir strategy, applied for antitumor strategies, is not new, but the form of delivery by VLP is,
VLP-CD40L—VLP decorated with CD40L, in combination with the VLP VSVg-TK: This new VLP-CD40L has two functions: it has tropism for tumor cells that express the CD40 receptor and has the ability to stimulate presenting cells at the tumor site, which will trigger the strengthening of the antitumor immune response. These presenting cells phagocyte tumor cell lysis debris, migrate to the lymphoid organ and interact with lymphocytes to mediate the antitumor response, or both.

The present VLPs are produced from retroviral capsids, meaning it is not necessary to produce any recombinant protein. VLP-CD40L is generated in final form, already containing CD40L on the surface.

Compared to the generation of dendritic cells containing CD40L, said VLP particles are much simpler to produce. In the case of cells, it is necessary to use the individual's own cells, modify them and return them to the individual. In the case of the present VLPs, they are administered to any individual, without the need of customizing cells.

The proposed VLPs are made with lentivirus-derived capsids, which have low immunogenicity. Hence, they can be administered systemically.

The VSVG TK VLPs were generated containing TK protein instead of TK RNA. The protein has a more direct action and is independent of the transcription that must occur from RNA. Said VLPs lack a viral genome, presenting a higher level of safety. No genetic material was carried in the VLP, only TK protein.

It is also claimed a kit for antitumoral therapy comprising: a VLP-derived from viral capsid decorated with VSV-g envelope glycoprotein and loaded with Thymidine Kinase (HSV-TK) from viral herpes; a VLP-derived from viral capsid decorated with CD40L; an antiviral drug, preferably ganciclovir, wherein the VLPs are preferably derived from a lentivirus capsid.

It is also claimed a method for antitumoral therapy comprising the administration of VLPs VSVg-TK, VLP CD40L and an antivirus, preferably ganciclovir. VSVg-TK VLP is administered locally. It has a high potential for internalization due to the known efficiency of the VSVg envelope, which has a broad spectrum of tropism for different cell types. Thus, the local administration of VLP VSVg-TK at the tumor site, and administration of the antivirus causes the death of tumor cells.

The administration of CD40L VLPs can be done locally or systemically, and it will act in synergy with the VLP-VSVg-TK, stimulating the activation of presenting cells that will phagocyte debris of dead tumor cells, which contain the necessary antigens for the antitumor immune system response. It plays a fundamental role in antigen presentation to lymphocytes, which can eliminate tumor cells.

This combined strategy has the potential to be benefited of the abscopal effect, that is, the ability to stimulate the immune system response in tumors distant from the tumor site. Furthermore, it is also possible to generate immunological memory, to enable long-term antitumor protection, with the potential to prevent relapses.

### Examples

The VLPs' production and the in vivo assays will be explained now on.

### Methodology

### Isolation and Activation of Primary CD4+ T Cells

Healthy isogenic C57BL/6 mice, aged 8-10 weeks, from the vivarium of the National Biosciences Laboratory (LNBio), were used to isolate CD4+ T lymphocytes from their spleens. Conventional CD4+ T lymphocytes were immunomagnetically isolated from splenocytes using negative selection (Magnasort Kit). The purity and yield of the separation protocol was verified by flow cytometry and was shown to be greater than 90%. These cells were activated for 24 hours with anti-CD3 (TONBO biosciences clone 145-2C11) and anti-CD28 (TONBO biosciences clone 37.51) antibodies at a concentration of 1 μg/ml.

RNA Extraction and cDNA Synthesis

Total RNA from activated CD4+ T cells and B16F10 cells was extracted with the Qiagen® RNeasy Mini Kit, following the manufacturer's instructions. The concentration of isolated RNAs was determined by absorbance analysis using the NanoDrop 2000/2000c equipment (Thermo Scientific). RNAs were stored at −80° C. The cDNAs were synthesized using the High Capacity RNA to cDNA™ kit Applied Biosystems™ and stored at −20° C.

### RT-PCR

The murine CD40L and HSV-TK genes were amplified by RT-PCR using the enzyme GoTaq® DNA Polymerase, from the cDNA of activated CD4+ T cells, isolated from the spleen of C57BL/6 mice, and from B16F10 cells. PCR reactions were performed with 10 ng of cDNA, 100 pmol of primers and 5 U/μl of GoTaq enzyme. The primers used in the amplification step were: GAPDH gene: F-AGATCGCTGTGAACGGATTTG (SEQ ID NO:1), R-TGTAGACCATGTAGTTGAGGTCA (SEQ ID NO:2); CD40L: F-GGGCCAGTCAGCATGATAGAAACA (SEQ ID NO:3), R-ACAGCGCACTGTTCAGAGTT (SEQ ID NO:4); and HSV-TK: F-AGAAAATGCCCACGCTACTG (SEQ ID NO:5), R-ATTGGCAAGCAGCCCGTAAA (SEQ ID NO:6); Amplification of the GAPDH gene was used as an internal control for each reaction.

Construction of the pcDNA-Hygro-CD40L Plasmid Expression Vector and the pCL-TK Viral Plasmid Vector

The pcDNA/Hygro-CD40L vector was constructed by inserting the murine CD40L gene into the pcDNA3.1/Hygro (+) expression plasmid vector (Invitrogen™). The CD40L sequence was amplified through the RT-PCR reaction, using cDNA from activated CD4+ T cells, isolated from the spleen of C57BL/6 mice. For amplification of the fragment, specific primers for murine CD40L were used. The PCR product was cloned into the PGEM®-T Easy vector and transformed into DH5a competent bacteria. The plasmid DNA was purified using the QIAGEN® Plasmid Midi Kit. The CD40L fragment was digested with the Notl restriction enzyme and subcloned into the pcDNA3.1/Hygro (+) vector, which was also cleaved with the same enzyme. The pCL-TK plasmid vector was constructed using the HSV-TK sequence taken from the PAL119-TK plasmid (Addgene #21911) and cloned into the pCLXSN vector, at the BamHl enzyme restriction site (NAVIAUX et al., 1996). Subsequently, the recombinant plasmids pcDNA/Hygro-CD40L and pCL-TK were transformed into thermocompetent DH5a bacteria, purified using the NucleoBond Xtra Midi Kit, sequenced and used in the production of a gene expression vector or retroviral vector for genetic modification, respectively.

### Establishment of the B16F10-TK Lineage

B16F10 cells were plated in 6-well plates, at a concentration of 1×105 cells per well, and transduced with the pCL-HSV-TK viral preparation, using MOI=1. Transduced cells were selected with 1600 μg/ml of the antibiotic Geneticin (G418). After selection of positive clones for the transgene, RT-PCR was performed to confirm the expression of the HSV-TK gene in the established cell line. HSV-TK protein expression was confirmed by cytotoxicity assay in the HSV-TK/GCV system.

### Analysis of the Bystander Effect

To investigate in vitro the bystander effect mediated by the HSV-TK/GCV system, B16F10 cells engineered with the GFP reporter gene were co-cultured with B16F10-TK cells in different proportions: 0:1, 1:9, 1:4, 2:3 and 4:1, and plated in a 96-well plate, at a concentration of 1×104 cells/well. Cells were treated with 50 μg/ml of GCV for 48 hours and the death of B16F10-GFP cells, mediated by the bystander effect, was assessed by GFP fluorescence intensity, measured by flow cytometry.

Labeling Tumor Cells with CFSE

B16F10 and B16F10-TK tumor cells were labeled with the lipophilic fluorescent dye CFSE. Cultured tumor cells were collected and centrifuged at 400 g×5 min at room temperature. The supernatant was discarded, and the pellet resuspended in PBS 5% serum. Cells were stained with 1.25 μM CFSE dye for 5 minutes at room temperature. Then, the cells were washed with PBS 5% serum, centrifuged at 400 g×5 minutes and cultured in D10 medium.

### Production of Viral Vectors in HEK 293T Cells

Viral vectors were generated by transient co-transfection of HEK 293T cells through calcium phosphate precipitation. Transfection efficiency was analyzed by flow cytometry. The viruses, present in the supernatant of transfected 293T cells, were collected every 24 hours, for 2 consecutive days. Viral preparations were titrated by flow cytometry. The production of viral vectors was carried out by the Viral Vectors Laboratory (LVV), located at the National Biosciences Laboratory (LNBio-CNPEM). Production protocols are based on Bajgelman et al (2003) and Strauss et al (2006).

### Cell Culture

The murine lines of melanoma B16F10, triple negative breast cancer 4T1 and fibroblasts 3T3 were maintained in D10 medium (DMEM (Dulbecco's Modified Eagle's Medium) supplemented with 1% penicillin/streptomycin, 1% glutamine and 10% serum fetal bovine (FBS). The cell lines, which were negative for contamination by Mycoplasma sp., were incubated at 37° C. in an atmosphere of 5% C02. Conventional CD4+ T cells were cultured in complete medium (RPMI (Roswell Park Memorial Institute), 1% penicillin/streptomycin, 10% FBS, glutamine, 1% HEPES, 1% sodium pyruvate, 1% non-essential amino acids and 50 μM β-mercaptoethanol and stored at 37° C. in a 5% C02 oven.

### Isolation of Bone Marrow Precursor Cells

Mice of the isogenic lineage C57BL/6 and Balb/c, healthy, 7-9 weeks old, coming, respectively, from the vivarium of the National Biosciences Laboratory (LNBio) and CEMIB (UNICAMP), were used for the isolation of cells precursors of bone marrow, from your femurs and your tibias. First, the tibias and femurs were extracted, cleaned with 70% alcohol and stored in ice-cold phosphate-buffered saline (PBS) 1×. Under sterile conditions, the bone heads were removed and bone marrow cells were collected by washing the interior of the bones with complete medium (RPMI (Roswell Park Memorial Institute), 1% penicillin/streptomycin, 10% FBS, glutamine, 1% HEPES, 1% sodium pyruvate, 1% non-essential amino acids and 50 μM β-mercaptoethanol), using a 1 ml syringe with a 24 G ¾″ 0.55×20 mm needle. Then, the cells were centrifuged at 400×g for 5 min, and the supernatant discarded. The cell pellet was resuspended in ACK RBC lysis buffer and incubated on ice for 5 minutes. Subsequently, the marrow cells were washed with 1×PBS to neutralize the effects of ACK and centrifuged at 400×g for 5 min. The supernatant was discarded, and the cells were resuspended in complete medium to generate dendritic cells.

### Differentiation and In Vitro Activation of Mouse BMDCs

To generate bone marrow-derived dendritic cells (BMDCs) from C57B6/L and Balb/C animals, bone marrow precursor cells were plated in a 24-well plate, at a concentration of 2×105 cells/well, and cultured in complete medium plus 20 μg/ml of GM-CSF (BioLegend) and IL-4 (BioLegend). The cells were maintained at 37° C. in an atmosphere of 5% CO2. Every 2 days, the culture medium containing non-adherent cells was removed and replaced with fresh complete medium supplemented with the same concentrations of GM-CSF and IL-4. After 7 days of differentiation, BMDCs were labeled with the Armenian Hamster IgG FITC (BioLegend, HTK888), IgG2aκ APC (eBioscience, eBR2), and IgG2aκ PE (BioLegend, MOPC-173), anti-CD11c FITC (BioLegend N418) antibodies., anti-CD86 APC (BioLegend, GL-1) and anti-MHCII PE (BioLegend, AF6-120.1) and characterized by flow cytometry.

### Production and Purification of VLPs

The production of VLPs was carried out by the Viral Vectors Laboratory (LVV), located at the National Biosciences Laboratory (LNBio/CNPEM). The different preparations containing the lentiviral VLPs CD40L and the negative control VLP-Hygro were generated by transient transfection in HEK 293T cells, as well as the pseudotyped lentiviral VLPs with VSV-G envelope: VSVG and VSVG-TK. To produce the VLPs, a plasmid encoding the pPax viral capsid, available at LVV, expression vectors engineered to encode the immunomodulator CD40L (pcDNA-Hygro-CD40L) and the HSV-TK gene (PAL119 TK—Adggene #21911) were used. and vector specific for the VSV-G viral envelope (pCMV-VSVG, available from LVV). For transfection, HEK 293T cells were cultured in D10 medium depleted of extracellular vesicles from fetal bovine serum. Supernatants from transfected 293T cells were collected every 24 hours for 3 consecutive days. Then, they were centrifuged at 400 g×5 min and filtered with a 0.22 μm diameter filter (Millipore) in order to remove cells, cellular debris and larger cellular vesicles. Subsequently, the VLPs were concentrated by ultracentrifugation at 110,000 g for 90 minutes at 4° C., with the aid of the SW32Ti rotor (Swinging-Bucket Rotor—Beckman Coulter). The pellets containing the particles were resuspended in PBS and stored at −80° C. for subsequent in vitro and in vivo characterization and functionality tests.

### Nanoparticle Tracking Analysis (NTA)

The biophysical analysis used to estimate the size and concentration of VLPs was performed using the NanoSight NS300 equipment (NanoSight, Amesbury, United Kingdom). As described in PALAMETA et al. (2022), the VLPs were diluted in 1 ml of 1×PBS to reach a concentration between 108-109 particles/ml. Next, the VLPs were introduced into a chamber containing a green laser with a wavelength of 532 nm. 5 videos of 60 seconds were captured per sample, at 18° C. Video acquisitions were performed using NTA software version 3.1 (Malvern Instruments, Worcestershire, UK) and camera adjustments were made appropriately to provide the correct focus of the particles. Both the size and concentration of VLPs were defined as an average of the 5 videos analyzed.

### Sample Preparation for Transmission Electronic Cryomicroscopy (Cryo-ME)

Sample preparation and analysis and acquisition of Crio-ME images were carried out by the electron microscopy facility at the National Nanosciences Laboratory/CNPEM. The samples were placed on copper grids for electron microscopy, with Lacey carbon film, 300 mesh (#01895-F, Ted Pella, USA). In the EasiGlow (I) equipment (Ted Pella, USA), the grids were treated with a load of 25 mA for 50 seconds. Subsequently, on the Vitrorobot Mark IV sample vitrification robot (Thermo, USA), the preparations of VLPs VSVG-TK and its control, VLP VSVG, and the VLPs CD40L and its control, VLP Higro, were applied to the grids, performing the step draining excess Blot time 3 and Blot force −3. Finally, the grids were frozen in liquid ethane and kept in liquid nitrogen until analyzed under a microscope.

Acquisition of Images from Crio-ME

The images were obtained using a transmission microscope model Talos Arctica (Thermo, USA), operated at 220 kV. For digital image acquisition, a Ceta 16M 4 k×4 k camera (Thermo, USA) was used. The images were analyzed using software for automated image collection.

### VLP Lysis

293T cells and VSVG and VSVG-TK VLP preparations were lysed with RIPA buffer (50 mM Tris (pH=7.4), 150 mM NaCl, 1% Triton X-100 and 1% sodium deoxycholate) supplemented with inhibitor of protease (PMSF, ThermoFisher) using a syringe equipped with a 30 G % ½″ 0.3×13 mm needle. The lysates were incubated on ice for 30 minutes and vortexed every 10 minutes. Then the cell preparations were centrifuged at 14000 rpm for 20 minutes at 4° C., while the VLPs were centrifuged under the same conditions for 4 hours. The supernatants resulting from centrifugation were stored at −80° C.

### Dot Blot

100 ug/ml of VLP lysate was immobilized on nitrocellulose membrane for 30 minutes. Then, the membrane was blocked for 1 hour in blocking solution containing TBS 2% BSA. Then, the membrane was incubated overnight at 4° C., with gentle agitation, in a 0.01% TBS tween solution and 5% skimmed milk (Molico) containing the anti-HSV-TK polyclonal antibody (produced by the Laboratory of Immunology and Engineering). LNBio-CNPEM antibodies) at a concentration of 1:500. Subsequently, the membrane was incubated with anti-mouse-HRP secondary antibody (Sigma-Aldrich A9004) and the presence of the protein was detected by a chemiluminescence reaction.

### Phenotypic Characterization of CD40L VLPs Using Bead-Based Flow Cytometry

CD40L VLPs were characterized for the presence of the CD40L protein, based on the inventor's protocols, previously described (PALAMETA et al., 2022; SEMIONATTO et al., 2020). To verify the presence of CD40L molecules on the surface of CD40L VLPs, 2×104 polystyrene beads were incubated overnight at 4° C. with 2 μg/ml of anti-CD40L biotin capture antibodies (Biolegend clone MR1). Then, the capture beads were blocked with PBS 2% BSA solution, for 5 hours at room temperature and gentle shaking, in order to block any non-specific binding sites that were free on the surface of the beads. Then, the capture beads were washed with 1 ml of PBS, centrifuged at 400 g for 5 minutes and incubated overnight with 1×1010 particles/ml of the CD40L VLP preparation. Subsequently, the VLPs bound to the beads were washed with 1×PBS, centrifuged for 5 minutes at 400 g and labeled with the anti-CD40L-APC antibody (eBioscience #17-1541-82). The presence of CD40L protein was analyzed by flow cytometry.

### Binding Assay of CD40L VLPs on Tumor Cells

Binding of CD40L VLPs to tumor cells was characterized using a flow cytometry assay. 1×105 B16F10 or 4T1 cells were incubated with 2×1010 particles/ml of CD40L VLPs or Higro VLP, used as control. Then, the cells were washed with PBS plus 5% serum and centrifuged at 400 g for 5 min. Subsequently, cells attached to VLPs were labeled with anti-CD40L-APC antibody (eBioscience #17-1541-82) for 30 minutes and CD40L expression measured by flow cytometry.

### In Vitro Cell Death Test

Parental B16F10 and 4T1 cells were plated at a concentration of 1×104 cells/well, in a 96-well plate with a flat bottom. Subsequently, the cells were transduced with the VLPs VSVG and VSVG-TK. At the same time, 50 μg/ml of GCV were added per well. The concentration of 50 μg/ml of GCV was determined through tests previously carried out by the inventors. Cells transduced with pseudotyped VLPs also received 8 μg/ml of polybrene, with the aim of increasing transduction efficiency. After 24 hours, the culture medium containing the VLPs and polybrene was removed and replaced with a new medium, plus 50 μg/ml of GCV. At the end of the 48-hour incubation period with GCV, the cytotoxic action of the VLPs was observed under an inverted light microscope. The cell culture supernatant was centrifuged at 400 g×5 min and stored at −80° C. for subsequent assays.

### MTT Test

The viability of B16F10 and 4T1 cells incubated with VSVG-TK VLPs was analyzed using the colorimetric assay of conversion of MTT into insoluble formazan crystals. Following the manufacturer's instructions (Invitrogen™), 12 mM MTT (3-(4,5-dimethylthiazol-2-yl)-2,5-diphenyltetrazolium bromide) was added to cells previously treated with the VLPs preparations VSVG and VSVG-TK and the prodrug GCV. For the cells to be labeled with MTT, they were kept at 37° C. for 2:00 hours. Then, the culture medium containing MTT was removed and replaced with 75 μL of dimethyl sulfoxide (DMSO). After 10 minutes of incubation at 37° C., the absorbance was measured with the ClarioStar equipment, at a wavelength of 540 nm. Absorbance data were analyzed using MARS software. Cell viability was expressed as a percentage of viable cells, following the formula: cell viability (%)=(experimental group/control group)×100%.

### Apoptosis Detection Assay

The apoptotic action of VSVG-HSV-TK VLPs was evaluated by flow cytometry through double labeling with annexin V-FITC and propidium iodide (PI) using the eBioscience™ Annexin V Apoptosis Detection Kit FITC. In this assay, B16F10 and 4T1 cells treated with VSVG-HSV-TK VLPs by the HSV-TK/GCV system were stained with annexin-V-FITC together with PI following the manufacturer's instructions. Tumor cells incubated with the VSVG-TK VLPs were collected, washed with PBS, resuspended in binding buffer, stained with annexin-V-FITC and the PI dye.

Assessment of the Maturation of BMDCs Treated with the Supernatant of Tumor Cells Killed by the HSV-TK/GCV System

Following 7 days of differentiation with GM-CSF and IL-4, BMDCs were incubated with the supernatant of 4T1 tumor cells treated with VSVG-TK VLP and ganciclovir for 48 hours. After this period, BMDCs were collected and characterized by flow cytometry, after labeling with the antibodies Armenian Hamster IgG FITC (BioLegend, HTK888), IgG2aκ APC (eBioscience, eBR2), and IgG2aκ PE (BioLegend, MOPC-173), anti-CD11c FITC (BioLegend N418), anti-CD86 APC (BioLegend, GL-1), anti-MHCII PE (BioLegend, AF6-120.1) and anti-CD40 (Tonbo FGK45). The supernatant of tumor cells treated with VSVG VLP was used as a negative control. BMDC incubated with LPS were used as a positive control for the experiment.

Activation Assay of BMDCs Treated with CD40L VLPs

After 7 days of differentiation with GM-CSF and IL-4, BMDCs were incubated with 1×1010 particles/ml of CD40L VLPs for 48 hours. Subsequently, DCs were collected and labeled with the antibodies Armenian Hamster IgG FITC (BioLegend, HTK888), IgG2aκ APC (eBioscience, eBR2), and IgG2aκ PE (BioLegend, MOPC-173), anti-CD11c FITC (BioLegend N418), anti-CD86 APC (BioLegend, GL-1), anti-MHCII PE (BioLegend, AF6-120.1) and anti-CD40 (Tonbo FGK45) and characterized by flow cytometry. BMDCs treated with LPS were used as a positive control, and BMDCs treated with Higro VLPs (VLPs constructed with a plasmid without the CD40L gene) were used as a negative control. The supernatant was stored at −80° C. for further analysis.

Apoptosis Detection Assay of Tumor Cells Treated with CD40L VLPs

B16F10 and 4T1 cells were plated at a concentration of 1×104/well in a 96-well plate and incubated with 2×1010 particles/ml of CD40L VLPs for 48 hours. Subsequently, the cells were collected and double labeled with Annexin-V-FITC and PI, following the steps described in item 3.14.3. Finally, the apoptotic action of CD40L VLPs on tumor cells was evaluated by flow cytometry.

### RT-qPCR

RT-qPCR analysis was performed in a 96-well plate and samples were tested in triplicates and gene expression quantifications were normalized to the expression of the endogenous control GAPDH. qPCR reactions were performed with 10 μl of iTaq Universal SYBR Green Supermix (BioRad®) (1×), 10 ηg of cDNA and 10 pmol/μl of primers for the BAX, FAS, CD40L, Casp3, Bcl-X and Ki67 genes. The primers used to amplify the genes of interest were: BAX: F-AGGATGCGTCCACCAAGAAGCT (SEQ ID NO:7), R-TCCGTGTCCACGTCAGCAATCA (SEQ ID NO:8); FAS: F-ATGCACACTCTGCGATGAAG (SEQ ID NO:9), R-CAGTGTTCACAGCCAGGAGA (SEQ ID NO:10); CD40L: F-GAACTGTGAGGAGATGAGAAGGC (SEQ ID NO:11), R-TGGCTTCGCTTACAACGTGTGC (SEQ ID NO:12); Casp3: F-GGAGTTCTGACTGGAAAGCCGAA (SEQ ID NO:13), R-CTTCTGGCAAGCCATCTCCTCA (SEQ ID NO:14); Bcl-X: F-GCCACCTATCTGAATGACCACC (SEQ ID NO:15), R-AGGAACCAGCGGTTGAACGGC (SEQ ID NO:16); and Ki67: F-GAGGAGAAACGCCAACCAAGAG (SEQ ID NO:17), R-TTTGTCCTCGGTGGCGTTATCC (SEQ ID NO:18). The relative expression of the genes was calculated based on the threshold cycle (Ct) of the exponential phase of amplification. The ΔCt value was measured by the difference between the Ct values of the genes of interest and GAPDH, using the equation: 2-Δ(ΔCt). Analyzes were performed using Applied Biosystems QuantiStudio5 software.

### Assessment of Tumor Volume of 4T1 Tumors in Balb/c Mice

Female mice of the Balb/c lineage, from the central vivarium of the State University of Campinas (CEMIB-UNICAMP), were maintained on a light/dark cycle every 12 hours and water and food ad libitum. 1×105 4T1 cells were injected subcutaneously into the left flank of the animals after anesthesia with ketamine and xylazine at concentrations of 100 mg/kg and 20 mg/kg, respectively, to ensure precise tumor implantation. Tumor growth was assessed daily using a digital caliper until the tumor volume reached a minimum size of approximately 20 mm3 (GORDON et al., 1992; LEINONEN et al., 2012). Then the animals were randomized and separated into different experimental groups, ensuring the number of at least 6 animals per group.

The treatment of tumors with VLPs followed the scheme of three doses injected intratumorally every two days (day 1, day 4 and day 7). Protamine sulfate was added to VLP preparations in order to increase the transduction efficiency of VSVG-TK VLPs in the tumor (BALAK et al., 2019; CORNETTA; ANDERSON, 1989). For eight consecutive days, starting from the first intratumoral injection, the prodrug ganciclovir was injected intraperitoneally at a concentration of 50 mg/kg (LEINONEN et al., 2012). Tumor volume was measured for 9 consecutive days, or until the tumors reached approximately 400 mm3, ensuring the animals' well-being. Tumor volume was measured according to the formula:

Tumor volume=((vertical measurement*horizontal measurement*diagonal measurement)*π/6).

At the end of the experiment, all animals were euthanized by an anesthetic overdose. The tumors and spleens were removed and used for histology and flow cytometry assays, respectively, in order to evaluate the immune cell profile in both. All animal experimentation procedures were approved by the Ethics Committee on the Use of Animals (CEUA) of the National Center for Research in Energy and Materials (CNPEM) contained in protocol 107.

### Tissue Preparation for Cryopreservation and Frozen Sectioning

The 4T1 cell tumors were embedded directly in the OCT (Optimum Cutting Temperature) compound and submerged in liquid isopentane at −90° C., so that they had a rapid and uniform initial freezing. Then, the samples were placed in liquid nitrogen at −159° C. and stored at −80° C. The tissues were sectioned at a thickness of 10 μm, and 3 serial sections were collected on each slide. The sections were carried out using Leica cryostat equipment, kept at −25° C.

### Immunofluorescence of Frozen Sections

The slides containing the frozen sections were thawed and kept moistened in PBS. Then, the sections were blocked with PBS 3% BSA for 30 minutes. Subsequently, the sections were washed 3 times with 1× PBS and incubated with a 1% BSA solution, containing the antibodies of interest in a 1:100 ratio, for 1 hour. The slides were washed with PBS to remove excess antibodies and analyzed under a Leica FS DM6 microscope.

### Spleen Cytometry

The spleens were macerated in a 70 μm cell strainer and treated with ACK to lyse the red blood cells. Subsequently, splenocytes were labeled with anti-CD4 (Biolegend GK1.5) and anti-CD8 (Tonbo 2.43) antibodies and analyzed by flow cytometry.

### Statistical Analyzes

All data were analyzed using GraphPrism 9.0. Statistical significance was assessed using the one-way ANOVA (analysis of variance) or two-way ANOVA, followed by Turkey's test for multiple comparisons. The two-way ANOVA test was performed to analyze tumor volume as a function of time. The results are expressed as a function of the mean+/−standard error of the mean that were normalized as a function of a control group. The statistical analysis with p<0.05 were considered to be significant.

### Results

The HSV-TK Enzyme Encoded by the Retroviral Vector pCL-HSV-TK is Stably Expressed in Tumor Cells

In order to understand how HSV-TK/GCV suicide gene therapy works in cancer cells, the first step was to establish a tumor line genetically modified with the HSV-TK enzyme. To achieve this, the HSV-TK transgene was inserted into B16F10 murine melanoma cells, using the pCL-TK retroviral vector.

The expression of the target gene within the transduced cells was identified by RT-PCR. Unlike parental B16F10 cells, the B16F10-TK line expresses the viral thymidine kinase gene in a stable manner, enabling its use in future investigations.

### B16F10-TK Cells Die in the Presence of GCV

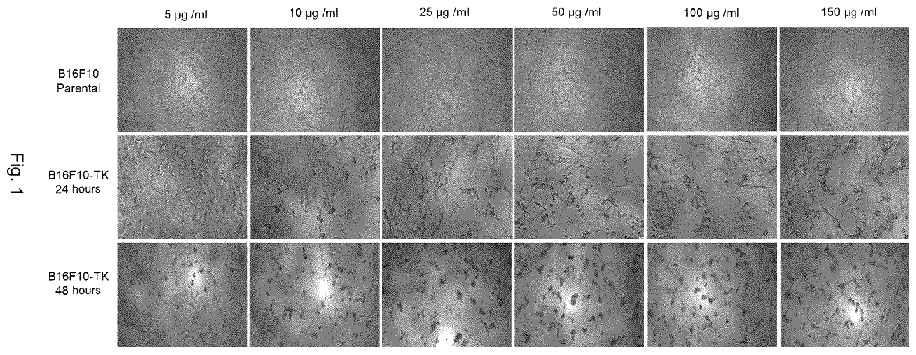

Many studies have already reported that tumor cells engineered with the HSV-TK gene undergo apoptosis when treated with the prodrug GCV (YI et al., 2018; MAJUMDAR et al., 2000; BROCKSTEDT et al., 2002). GCV is specifically metabolized into its active form by viral thymidine kinase, transforming into a toxic metabolite that inhibits DNA synthesis. In view of this, after successfully constructing the pCL-TK retroviral vector and establishing the B16F10-TK lineage, it was analyzed whether engineered tumor cells suffered tumor death by the HSV-TK/GCV system. The results demonstrated that B16F10-TK cells incubated with GCV suffered significant tumor death compared to parental B16F10 cells, used as a negative control. It was also demonstrated that this cytotoxic effect is dependent on the increasing concentration and time of action of GCV, as shown in FIG. 1.

FIG. 1 shows the death of tumor cells engineered with the viral enzyme thymidine kinase by the HSV-TK/GCV system. B16F10-TK cells treated with increasing concentrations of ganciclovir (5, 10, 25, 50, 100 and 150 μg/ml) showed cell death in the first 24 hours. After 48 hours of treatment with ganciclovir, the cytotoxic effect was even more pronounced, showing that 10 μg/ml of the prodrug was sufficient to promote a significant reduction in the number of tumor cells.

### The HSV-TK/GCV System Suppresses Tumor Cell Proliferation

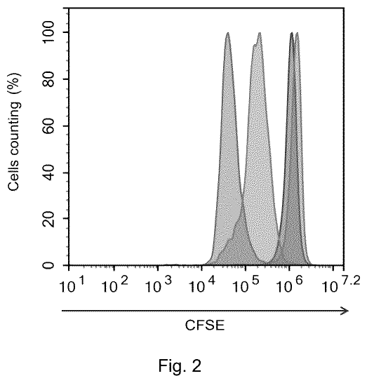

In order to explore the death mechanisms through which the HSVTK/GCV system exerts its cytotoxic function, it was performed an in vitro assay to evaluate the antiproliferative activity of the HSV-TK enzyme in combination with the GCV prodrug. In this experiment, the proliferation of B16F10-TK cells was determined by decreasing the fluorescence intensity of the vital dye Carboxyfluorescein Succinimidyl Ester (CFSE). As CFSE stains the cell plasma membrane, its fluorescence intensity decreases as cells proliferate. According to the results, a broad decrease in the proliferation of B16F10-TK cells cultured for 24 hours with GCV was identified. However, parental B16F10 cells treated with the prodrug ganciclovir for the same period did not exhibit a reduction in their proliferative capacity, as shown in FIG. 2.

FIG. 2 shows the HSV-TK/GCV system inhibiting the proliferation of B16F10-TK cells. B16F10-TK cells showed inhibition of proliferation after being treated with the prodrug ganciclovir for 24 hours (blue peak). Differently, parental B16F10 cells continued to proliferate even when treated with ganciclovir (green peak). The red and gray peaks represent day zero of labeling and treatment of parental B16F10 and B16F10-TK cells with CFSE and gancilcovir, respectively. CFSE expression was measured by flow cytometry. Representative histogram of two independent experiments.

### Bystander Effect Amplifies the Cytotoxicity of the HSV-TK/GCV System

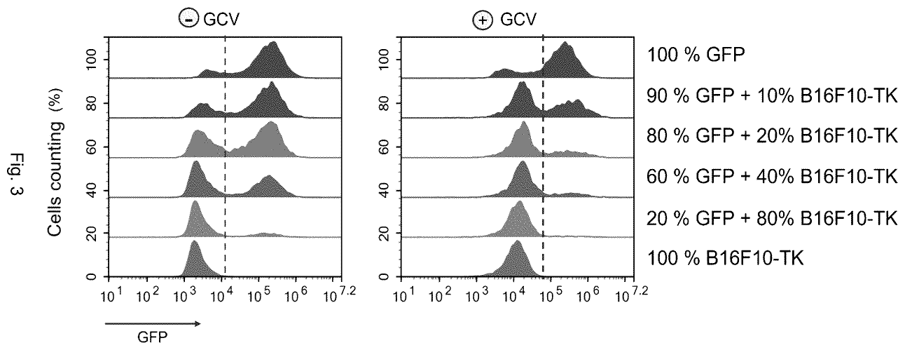

An important property of HSV-TK suicide gene therapy in combination with the GCV prodrug is the bystander effect. In this context, tumor cells that express the HSV-TK enzyme, when exposed to GCV, cause the death of adjacent cells that do not express the viral thymidine kinase enzyme. This process of death extended to adjacent cells occurs through the transfer of the toxic metabolite triphosphate ganciclovir through cell junctions (gap junctions), expanding the effects of tumor death. Thus, to investigate the bystander effect mediated by the HSV-TK/GCV system in vitro, B16F10-TK cells were co-cultured with B16F10-GFP cells in different proportions and treated with GCV. The death of B16F10-GFP cells, mediated by the bystander effect, was assessed by measuring the intensity of GFP fluorescence by flow cytometry, as shown in FIG. 3. The results indicate a decrease in GFP expression under conditions in which co-cultures were incubated with GCV. It is interesting to note that even in smaller quantities of B16F10-TK cells, there was a potential to reduce the number of GFP-positive tumor cells.

FIG. 3 shows a co-culture assay to evaluate the bystander effect of the HSV-TK/GCV system on cancer cells. B16F10-TK cells were co-cultured in different proportions (0:10, 1:9, 1:4, 2:3, 4:1 and 10:0), with B16F10 cells genetically modified with the GFP reporter gene. Ganciclovir treatment promoted a decrease in GFP intensity when B16F10-GFP cells were co-cultured with B16F10-TK, demonstrating the bystander effect of the HSV-TK/GCV system. Lower proportions of B16F10-TK cells were sufficient to lead to a reduction in GFP expression (1:9 and 1:4). GFP intensity was analyzed by flow cytometry. Representative histograms from an independent experiment.

### Tumor Cells Killed by the HSV-TK/GCV System Induce the Activation of BMDCs.

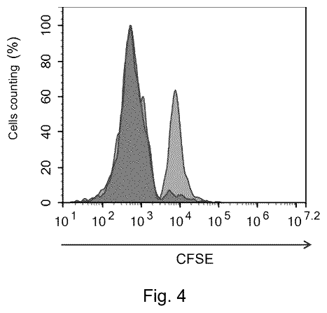

In addition to the potent tumoricidal activity triggered by the bystander effect in the tumor microenvironment, it was sought to examine the systemic effects generated by the HSV-TK/GCV system, exploring its role in activating antitumor immune responses. Data in the literature demonstrate that dead cancer cells produce a diverse range of tumor antigens and DAMPs, which would be capable of activating dendritic cells (VILE et al., 1997). The first step was to certify the presence of dead cells or cell fragments in the supernatant of B16F10-TK cells killed by the HSV-TK/GCV system. To do this, cells were initially stained with CFSE, subsequently treating them with GCV. The CFSE dye impregnates the cell, and in the case of cell death, the resulting debris is labeled with CFSE, which can be detected by fluorescence analysis. Next, it was incubated the culture medium (supernatant) from dead cells with a preparation containing micrometric polystyrene beads, which have a high affinity for binding to proteins, in a non-specific way. Based on the results obtained, it was observed that the beads incubated with the supernatant of B16F10-TK cells previously stained with CFSE emitted high intensity of fluorescence, while those incubated with the supernatant of parental B16F10 cells did not show a significant increase in fluorescence after being analyzed by cytometry flow, as shown in FIG. 4.

FIG. 4 shows that the tumor cells killed by the HSV-TK/GCV system release cellular debris and tumor antigens into the culture medium. Polystyrene beads incubated with the supernatant of B16F10-TK cells stained with CFSE and cultured with ganciclovir for 24 hours showed increased CFSE expression (green peak shifted to the right). The supernatant from parental B16F10 cells, labeled with CFSE and treated with GCV, was adsorbed to the beads and used as a negative control (red peak). CFSE fluorescence intensity was measured by flow cytometry and the data was analyzed using the FCS Express 5 software. x-axis represents the mean fluorescence intensity (MFI) on a logarithmic scale and the y-axis represents the percentage of cell number. Representative histogram of two independent experiments.

Next, an in vitro assay was performed in which the supernatant collected from B16F10-TK cells killed by the action of GCV was combined with dendritic cells differentiated from the bone marrow of mice and evaluated the activation of BMDCs through increased protein expression. MHC II and the costimulatory molecule CD86. As shown in FIG. 5, the supernatant of B16F10-TK cells killed by the HSV-TK/GCV system induced an increase in the expression of MHC II and CD86 markers in BMDCs, compared to the supernatant of parental cells.

FIG. 5 shows the bone marrow-derived dendritic cells activate in the presence of fragments of tumor cells killed by the HSV-TK/GCV system. Dendritic cells differentiated from mouse bone marrow were incubated with a fragment of B16F10-TK cells, present in the cell culture medium plus GCV. Activation was analyzed using flow cytometry, quantifying the increase in expression of MHC II and CD86 molecules, compared to differentiated cells without activation stimulus. CD11c represents a specific marker of dendritic cells.

In Vitro Assays for Phenotypic Characterization of Target-Directed VLPs Engineered with the Immunomodulator CD40L and the Suicide Enzyme HSV-TK.

After understanding how the HSV-TK/GCV system works in a model of tumor cells that expressed the HSV-TK enzyme, it was verified the immunomodulatory and tumoricidal functionality of VLPs engineered with the CD40L protein (CD40L VLP) and the HSV-TK (VSVG-TK), respectively. Using the VLP isolation, clarification, and purification methodologies described in Materials and Methods, VLP preparations were characterized using a series of biophysical methods. Using flow cytometry assays, the existence of the CD40L molecule on the surface of CD40L VLPs was identified. The presence of the HSV-TK protein inside the VSVG-TK VLPs was detected using a Dot-blot assay using the lysate of these particles. The nanoparticle tracking technique (NTA) and transmission electron cryomicroscopy (Cryo-ME) were used to estimate the size and concentration of different VLP preparations and their morphological aspects.

VLPs Derived from Lentiviral Capsids Exhibit the Same Size Distribution and Characteristic Morphology of Parental Particles

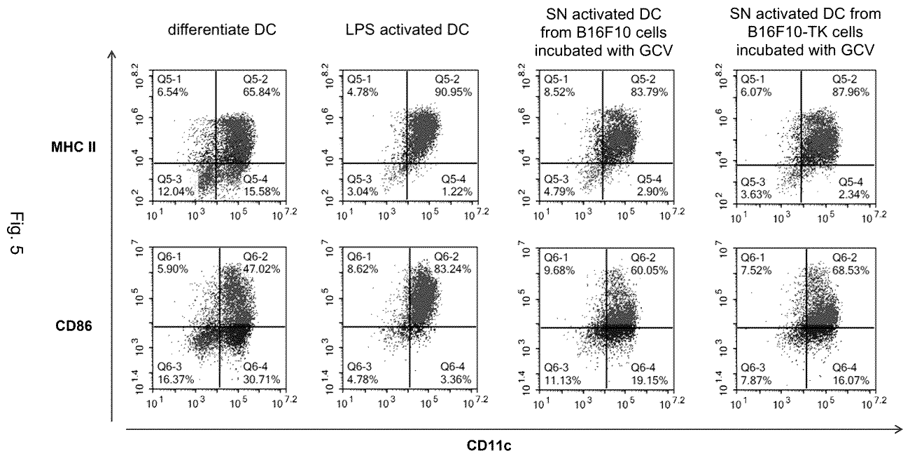

It was evaluated whether the different VLP preparations, constructed from lentiviral capsids, resembled the size and morphology of parental lentiviral particles, which have an average size of around 100 ηm, a spherical structure, with a highly electron-dense central area, and a conical-shaped capsid centered inside the particle (MARTIN et al., 2016; GANSER et al., 1999). In the case of a VSV-G envelope, it would be expected to verify the presence of characteristic spikes (SCHODERBOECK et al., 2015). Initially, the concentration and size of the nanoparticles were measured by NTA analysis. This technique allows the measurement of the size distribution and concentration of nanoparticles in liquid suspension, using the properties of light scattering and Brownian motion. According to the results shown in FIG. 6, the average diameter size of the particles engineered with HSV-TK and decorated with CD40L was 153.5+/−2.5 nm and 153 nm+/−1.4 nm, respectively. While the concentration of VSVG-TK VLPs preparations 1.59E+12+/−7.2E+10 particles/ml and 1.37E+12+/−5.73E+10 particles/ml for CD40L VLPs.

FIG. 6 shows the measurement of the concentration and average size of VLPs VSVG-TK and CD40L preparations using the NTA equipment. In histograms A and B it can be observed the average concentration in particles/ml (y axis) by the average size in nm (x axis) of the VLP VSVG-TK and VLP CD40L preparations, respectively. Representative image from at least three independent experiments.

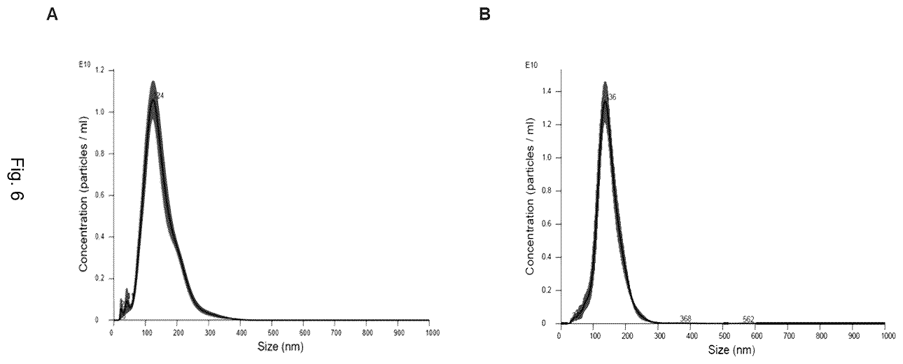

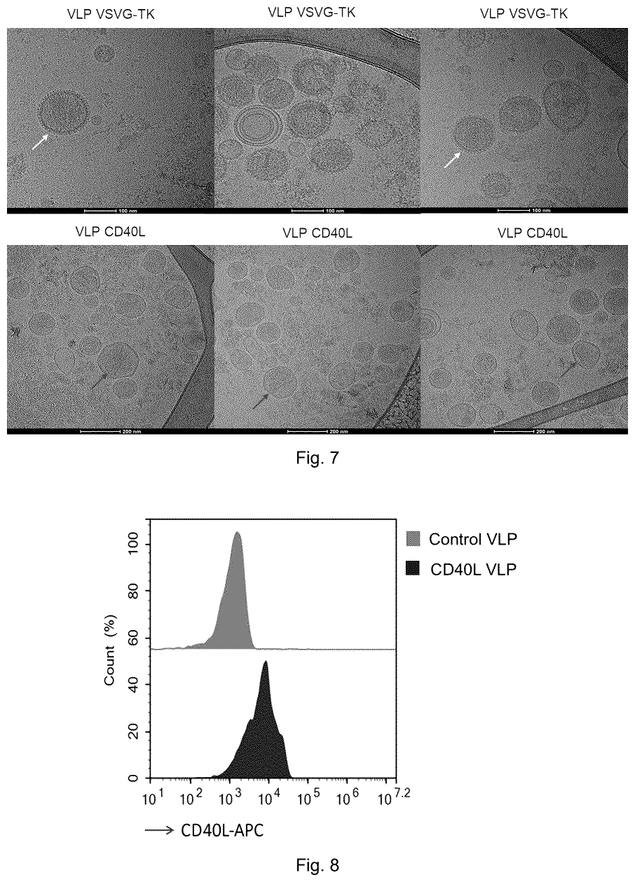

In addition to determining the diameter and average concentration of VLP preparations, VLPs VSVG-TK and CD40L were analyzed morphologically using Crio-ME. The Crio-ME images confirmed the typical morphology of lentivirus-derived particles: spherical structure, with a highly electron-dense central area, and a conical-shaped capsid centered inside the particle (MARTIN et al., 2016; GANSER et al., 1999), as shown in FIG. 7. On the membrane of VSVG-TK VLPs, it was also possible to visualize glycoprotein spikes originating from the VSV-G envelope, also shown in FIG. 7. Thus, in addition to the structural analysis of the nanoparticles, the Cryo-EM images confirmed the presence of the particles of interest in the VLP VSVG-TK and VLP CD40L preparations.

FIG. 7 shows a transmission electron cryomicroscopy (Cryo-EM) for morphological analysis of the VLP VSVG-TK and VLP CD40L preparation. TK-VLPs appear as spherical and electron-dense structures, surrounded by spikes referring to vesicular stomatitis virus envelope glycoprotein G (VSV-G) (open arrows). Inside the CD40L VLPs, it is possible to observe the centralized conical capsid (red arrows) characteristic of lentiviral particles. Scale bar represents 100 nm for VSVG-TK VLPs and 200 nm for CD40L VLPs.

VLPs Derived from Lentiviral Capsids Carry the Costimulatory Protein CD40L on their Surface.

The present studies have already reported that biological nanoparticles can be engineered to anchor TNFSF ligands, such as 4-1 BBL and OX40L, and molecules that offer tropism to tumor cells (PALAMETA et al., 2022; SEMIONATTO et al., 2020). The goal was to demonstrate the versatility of target-directed VLPs as a delivery system for different immunomodulatory proteins. First, the recombinant plasmid pcDNA-hygro-CD40L was constructed and CD40L VLPs was generated through co-transfection of HEK 293T cells.

After producing, isolating and purifying CD40L VLPs through centrifugation, filtration and ultracentrifugation steps, the presence of the CD40 ligand on the VLP membrane by bead-based flow cytometry was confirmed, as shown in FIG. 8.

FIG. 8 shows the characterization of CD40L VLPs. Preparations of CD40L VLPs were incubated with anti-CD40L capture beads. CD40L VLPs bound to capture beads were labeled with an anti-CD40L-APC antibody, and CD40L expression was analyzed by flow cytometry. Beads incubated with VLPs constructed only with the pcDNA 3.1/Higro (+) plasmid, without the insertion of the CD40L transgene (control VLP), served as experiment controls. Preparations of CD40L VLPs stained with the anti-CD40L-APC antibody showed a significant increase in fluorescence intensity (blue peak) when compared to control VLPs (gray peak). Representative image from three independent experiments.

VLPs Derived from Lentiviruses Pseudotyped with the VSVG Envelope May Harbor HSV-TK

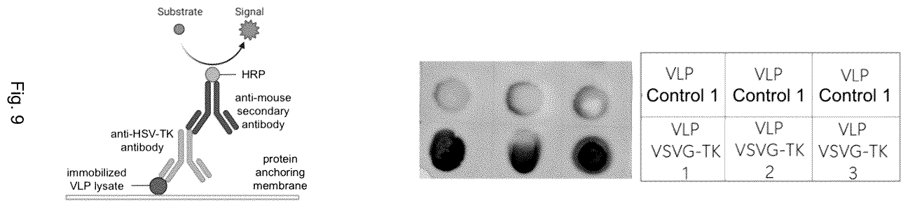

In addition to the ability to transport immunomodulators and molecules that target the tumor site, it was investigated whether VLPs derived from lentiviruses pseudotyped with the VSV-G envelope were able to carry the protein encoded by the HSV-TK gene. The first step was to identify the presence of the HSV-TK enzyme inside the VSVG-TK nanoparticles. For this purpose, the VLP lysate was immobilized on a nitrocellulose membrane and detected the presence of the protein of interest using the Dot Blot assay. The primary murine anti-HSV-TK antibody used in this assay was produced at the Laboratory of Immunology and Antibody Engineering, at LNBio-CNPEM. The production and obtaining of this polyclonal antibody was essential to evaluate the ability of VSVG-TK VLPs to transport the viral thymidine kinase within them. After adding the VSVG-TK VLP lysate to the membrane, colorimetric detection of the protein of interest was carried out. As shown in FIG. 9, VSVG-TK VLPs are capable of carrying the HSV-TK enzyme inside them. In contrast, the HSV-TK enzyme was not detected in the lysate of VSVG VLPs, used as a negative control for the experiment.

FIG. 9 shows that the VSVG-TK VLPs carry the HSV-TK suicide enzyme within them. Dot blot assay for colorimetric detection of the HSV-TK enzyme present in the VSVG-TK VLP lysate. VLP lysates were labeled with murine anti-HSV-TK primary antibody and HRP-conjugated secondary antibody. The lysate from the VSVG VLPs preparation was used as a negative control. This figure is a representative image from three independent experiments.

VSVG-TK VLPs Carry the Protein Encoded by the HSV-TK Suicide Gene, but not the mRNA Encoded by the Same Gene

Although the efficiency of VSVG-TK VLPs in delivering the viral thymidine kinase was proven, it was also investigated whether VSVG-TK VLPs could package the mRNA encoded by the HSV-TK suicide gene plasmid vector, transfected into the packaging cell. To achieve this, it was transduced B16F10 cells with the recombinant viral vector pCL-TK, which encoded the HSV-TK gene, or with VSVGTK VLPs. The pCL-TK vector presents a “psi” packaging sequence, which is transcribed upstream of the RNA, directing the molecule towards packaging. In the case of VLP, the expression vector is pall 19-HSV-TK, which does not encode the “psi” packaging sequence, and therefore, it was not expected to verify RNA related to TK packaged in the particle. As viral vectors carry the viral genome, containing the “psi” sequence, packaged inside, tumor cells genetically modified with these particles should express the mRNA corresponding to the HSV-TK gene, resulting from the integration of the virus genome into the cellular DNA. However, cancer cells transduced with VSVG-TK VLPs, which lack a viral genome, should not express the transcript related to the HSV-TK gene, which does not have a “psi” packaging sequence, unless these particles have Even so, the messenger RNA coding for the HSV-TK enzyme was packaged, which could then be delivered to the target cell. Thus, mRNA from tumor cells incubated with pCL-TK viral particles and VSVG-TK VLPs were extracted and processed for RT-PCR, in order to evaluate the presence of HSV-TK mRNA in the transduced cells. The results indicated that B16F10 cells incubated with VSVG-TK VLPs did not express the HSV-TK gene mRNA, unlike cells genetically modified with the pCL-TK viral vector. Furthermore, it was demonstrated that viral vectors produced with envelopes containing different infective potential (amphotropic or pantotropic (VSV-G)) did not interfere with the packaging and transport of viral mRNA into the tumor cell. It also highlighted the need for an intact viral particle to deliver mRNA encoded by the HSV-TK gene into tumor cells. It was observed that B16F10 cells transduced with supernatant from 293T cells transfected only with the plasmids that contained the HSV-TK enzyme sequence, but not with the plasmids that encoded the viral envelope and capsid, did not express the mRNA of the viral thymidine kinase gene in the interior.

### VSVG-TK VLPs Cause Tumor Cell Death In Vitro Through Decreased Cell Viability and Apoptosis Mechanisms

It was verified that VSVG-TK VLPs are internalized by cancer cells, causing them to die by the HSV-TK/GCV system. Furthermore, it was observed that the cytotoxic action of these VLPs in association with the prodrug ganciclovir is based on the decrease in viability and induction of cell apoptosis.

### The VSVG Envelope is Required for the Internalization of Lentiviral VLPs by Tumor Cells

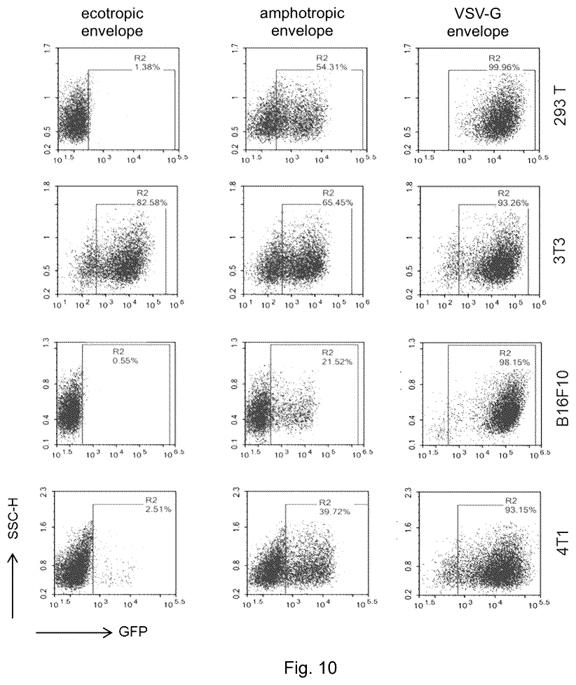

After verifying the possibility of carrying HSV-TK through VLPs pseudotyped with a VSV-G envelope, the use of other types of envelope that are commonly used in retroviral preparations was tested, such as the ecotropic envelope, which is described in the literature as presenting tropism for murine cells and the amphotropic envelope, which presents tropism for murine and human cells (NAVIAUX et al., 1996b). To this end, it was performed an assay in which murine 3T3, B16F10 and 4T1 cells and human 293T cells were transduced with recombinant lentiviral preparations containing a constitutive expression cassette of the GFP reporter gene and the different viral envelopes. As seen in FIG. 10, the amphotropic envelope, which has tropism for mammalian cells, and the pantotropic VSV-G envelope, which infects a wide variety of cell types from different species, were efficient in transducing murine 3T3, B16F10 and 4T1 cells. However, 3T3 cells transduced with viruses containing an ecotropic envelope showed high expression of the GFP reporter gene, unlike B16F10 and 4T1 tumor cells, which showed a low percentage of GFP-positive cells.

Based on the results obtained, the VSVG-TK VLPs pseudotyped with the VSV-G envelope were considered more advantageous. In addition to the broad tropism of this envelope for the host cell, it also confers high physical stability to the particles, allowing them to be concentrated through ultracentrifugation at high speeds and have better yields (ICHIM; WELLS, 2011).

FIG. 10 shows a transduction test with lentiviral preparations encoding GFP, produced with different envelopes. The figure shows flow cytometry panels resulting from transduction with pseudotyped preparations with three different envelopes, with four different cell lines. Lentiviral vectors produced with an ecotropic envelope are not internalized by B16F10e 4T1 cells. x-axis represents the mean fluorescence intensity (MIF) on a logarithmic scale. Representative image from 2 independent experiments. GFP: Green Fluorescent Protein; SSC: Side scatter.

### VSVG-TK VLPs Cause Tumor Cell Death Via the HSVTK/GCV System

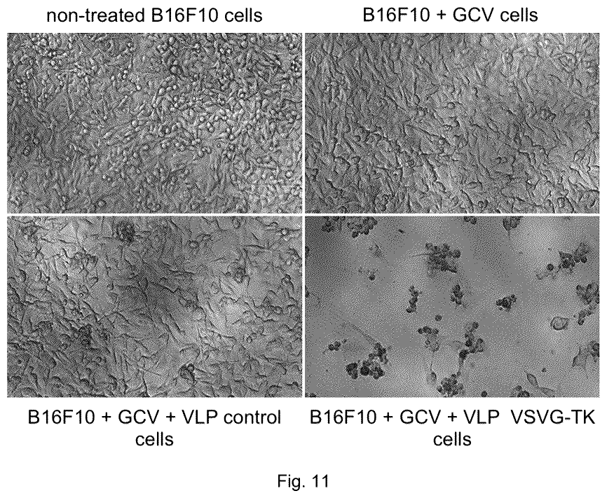

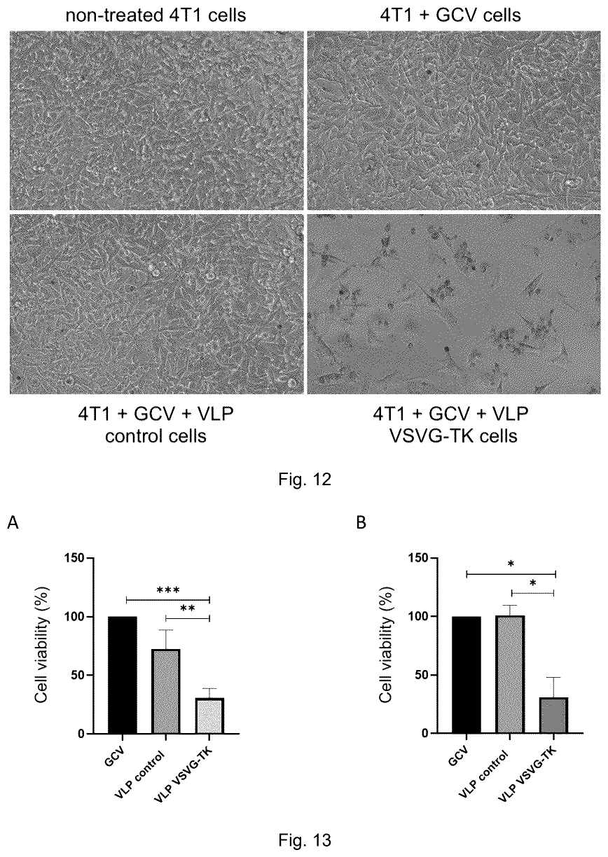

The cytotoxic activity of VSVG-TK VLPs was evaluated for their ability to induce the death of B16F10 and 4T1 tumor cells in vitro. In this experiment, the cytotoxic effect of VSVG-TK particles together with the GCV prodrug was estimated by visually reducing the number of tumor cells under an inverted light microscope. As shown in FIGS. 11 and 12, it was found that the GCV-associated VSVG-TK VLPs were efficient in significantly decreasing the number of tumor cells derived from B16F10 murine melanoma, as shown in FIG. 11, and tumor cells derived from 4T1 murine breast cancer, as shown in FIG. 12, highlighting the cytotoxic potential of these particles by the HSV-TK/GCV system.

FIG. 11 shows that the VSVG-TK VLPs drive the death of murine melanoma cells by the HSV-TK/GCV system. Inverted light microscope analysis of the death of tumor cells treated with VLPs VSVG-TK and the prodrug ganciclovir. B16F10 cells underwent significant tumor death compared to cells treated with control VSVG VLPs and ganciclovir. Controls: parental cells (mock); parental B16F10 cells cultured with GCV (GCV); parental B16F10 cells cultured with VSVG and GCV VLPs.

FIG. 12 shows that the VSVG-TK VLPs drive the death of murine triple-negative breast cancer cells by the HSV-TK/GCV system. Inverted light microscope analysis of the death of tumor cells treated with VLPs VSVG-TK and the prodrug ganciclovir. 4T1 cells underwent significant tumor death compared to cells treated with control VSVG VLPs and ganciclovir. Controls: parental cells (mock); parental 4T1 cells cultured with GCV (GCV); parental 4T1 cells cultured with VSVG and GCV VLPs.

### VSVG-TK VLPs Decrease Cell Viability and Induce Apoptosis of Tumor Cells by the HCV-TK/GCV System

After finding that tumor cells transduced with VSVG-TK VLPs were sensitive to GCV, the antitumor mechanisms mediated by the HSV-TK/GCV system were evaluated. First, it was detected cell viability through a colorimetric MTT assay, in which viable cells reduce the MTT compound (yellow) to insoluble formazan crystals (purple). When tumor cells were incubated with VSVG-TK VLPs and treated with the GCV prodrug, there was a significant reduction in cell viability, as shown in FIGS. 13A and 13B. Likewise, it was observed that tumor cells treated with VSVG-TK VLPs showed a significant decrease in cell viability compared to the condition treated with the control VLPs preparation. There was also a small decrease in cell viability after the tumor cells were treated with the control VLPs compared to the GCV control. This effect can be attributed to the cytotoxic properties inherent to the VSV-G envelope (YANG et al., 1995).

FIGS. 13A and 13B show that the VSVG-TK VLPs in association with the prodrug ganciclovir (GCV) promote a decrease in the viability of tumor cells. MTT assay to analyze the viability of B16F10 (A) and 4T1 (B) tumor cells incubated with the indicated VLPs, in the presence of GCV. Cells treated with VSVG-TK VLPs showed a significant decrease in cell viability compared to conditions in which tumor cells were cultured with GCV alone or treated with the control VLP preparation (VSVG VLP). Although VSVG VLPs lead to a certain decrease in the viability of B16F10 cells, this cytotoxic function may be associated with the cytotoxic profile of the VSVG protein. Representative graphs of 3 independent experiments. The one-way ANOVA test followed by the Tukey test were used for statistical analysis, considering the values of ***p<0.005 and *p<0.05.

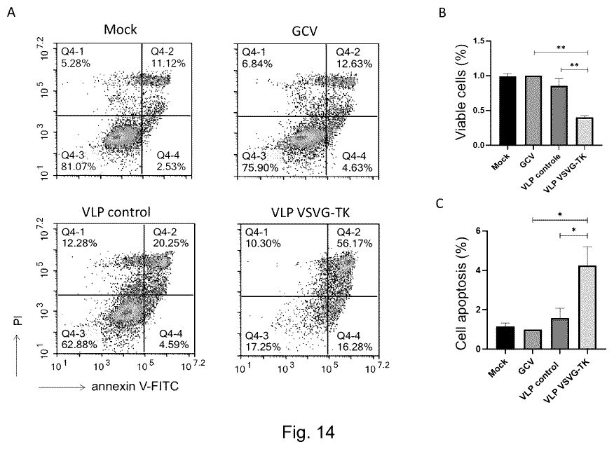

Therefore, knowing that VSVG-TK VLPs impact the viability of tumor cells in vitro, a cytometry assay with annexin-V/PI was performed to determine the apoptosis of B16F10 tumor cells transduced with VSVG-TK VLPs by flow cytometry. The annexin-V/PI assay suggested that the death of most tumor cells by the HSV-TK/GCV system is triggered by cellular apoptosis, observing an increase in double annexin-V/PI labeling in cells incubated with VLP VSVG-TK in the presence of the prodrug ganciclovir, compared to cells treated with control VLPs, as shown in FIG. 14.

FIG. 14 shows that the VSVG-TK VLPs lead to tumor cell apoptosis by the HSV-TK/GCV system. (A) Annexin-V-FITC and PI double staining assay. Flow cytometry of B16F10 cells labeled with annexin-V-FITC and PI after being treated with the preparation of VSVG-TK VLPs and VSVG VLPs, used as control. B16F10 tumor cells treated with VSVG-TK VLPs showed a significant increase in the number of apoptotic cells, labeled with both annexin-V-FITC and PI. (B) Reduction in the number of viable tumor cells after treatment with VSVG-TK VLPs in combination with ganciclovir. (C) Tumor cells treated with the VLP preparation VSVG-TK and ganciclovir show a high percentage of apoptosis. Controls: mock (B16F10 cells cultured with D10 medium); GCV (B16F10 cells cultured with D10 plus GCV); VSVG VLP (cells cultured with medium containing GCV and VSVG VLPs). Data analysis was performed using the GraphPad Prism 9.0 program, using the one-way ANOVA statistical test followed by the Tukey test. Values *p<0.05 and *p<0.005. Representative image from two independent experiments.

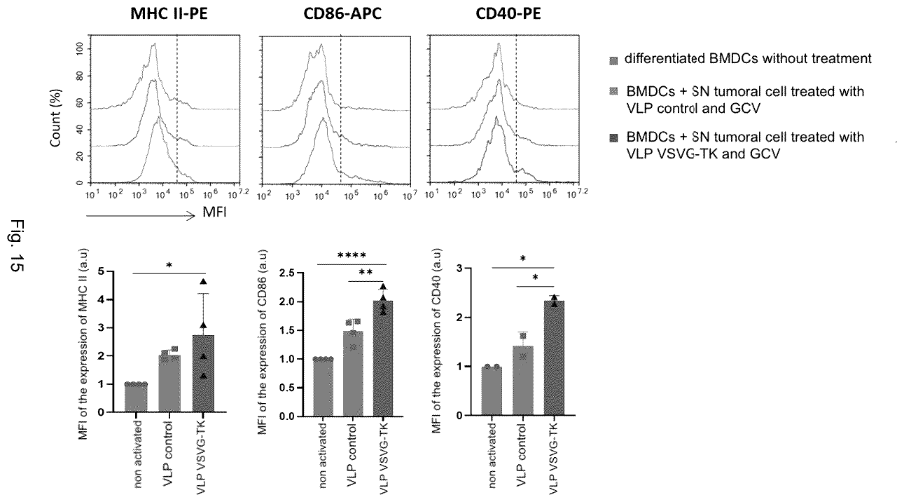

The death of tumor cells by the HSV-TK/GCV system, mediated by VSVG-TK VLPs, leads to the release of cell fragments capable of activating BMDCs. After observing the cytotoxic potential of VSVG-TK VLPs, it was investigated whether cellular apoptosis mediated by these particles, together with the prodrug ganciclovir, were capable of releasing cellular fragments that could act in the maturation of BMDCs. To achieve this, BMDCs were incubated with the supernatant of 4T1 tumor cells killed by the HSV-TK/GCV system, mediated by the action of VSVG-TK VLPs. After 48 hours of incubation, it was observed by flow cytometry the increased expression of the markers CD86 and CD40 on the surface of BMDCs, treated with the supernatant of 4T1 tumor cells that were incubated with the VLPs VSVG-TK and ganciclovir, as shown in FIG. 15. Differently, the expression of CD86 and CD40 in BMDCs incubated with the supernatant of tumor cells treated with control VLPs did not show a considerable increase in the mean fluorescence intensity. Although the expression of MHC II protein in BMDCs incubated with supernatant from cells killed by the HSV-TK/GCV system was not as pronounced in relation to other markers, the presence of MHC II protein was also greater in these cells compared to those cultured with the control supernatant. These results demonstrate that, in addition to the cytotoxic action of VSVG-TK VLPs on tumor cells, an additive effect on BMDCs was observed, triggering the activation of these cells. Interestingly, these data corroborate previously demonstrated findings, in which tumor cells engineered with the HSV-TK suicide gene and treated with GCV released cellular fragments capable of activating BMDCs.

FIG. 15 shows that the supernatant from dead tumor cells after treatment with VSVG-TK VLPs and ganciclovir releases cellular fragments that activate BMDCs. Representative image from an independent experiment. Graphs of mean fluorescence intensity. MFI: Mean Fluorescence Intensity.

### CD40L VLPs Induce Bone Marrow-Derived Dendritic Cell Maturation and Cancer Cell Apoptosis Through CD40:CD40L Signaling

Aiming to enhance antitumor immune responses mediated by VSVG-TK VLPs in association with the GCV prodrug, VLPs decorated with the co-stimulatory protein CD40L were developed, in order to intensify the antitumor activity of dendritic cells and promote apoptosis of dendritic cells. cancer in an additive manner to the cytotoxic and immunomodulatory effects of the HSV-TK/GCV system.

### CD40L VLPs Increase the Expression of Activation Markers on the Surface of BMDCs

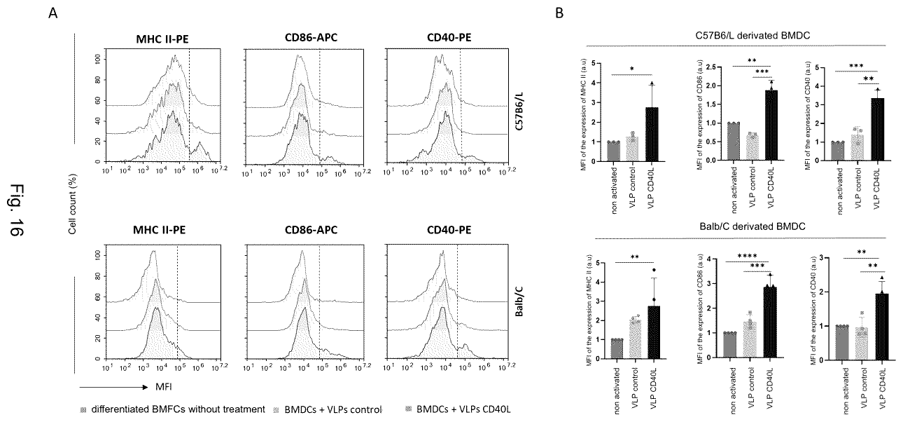

CD40:CD40L signaling is essential for dendritic cells to become able to activate CD8+ T lymphocytes, generating potent antitumor immune responses. In view of this, it was evaluated whether VLPs decorated with the CD40L protein were efficient in inducing the maturation of bone marrow-derived dendritic cells. For this purpose, CD40L VLPs were incubated with BMDCs for 48 hours. Next, the increased expression of MHC II activation markers was analyzed, CD86 and CD40 on the surface of dendritic cells. The presence of the MHC II molecule and the costimulatory proteins CD86 and CD40 increased considerably in BMDCs treated with CD40L VLPs, compared to those that were cultured with control VLPs, free of the CD40L protein in their membrane, as shown in FIG. 16.

FIG. 16 shows that the CD40L-engineered VLPs induce activation of BMDCs. (A) Representative histograms of the increase in the mean fluorescence intensity of MHC II, CD86 and CD40 proteins in BMDCs derived from C57B6/L and Balb/c animals, after 48 hours of treatment with CD40L VLPs. Unlike BMDCs treated with control VLPs (peak in orange), which showed less difference in the expression of activation markers compared to those that did not receive any treatment (peak gray), BMDCs incubated with CD40L VLPs (peak in blue) exhibited an increase of the expression of the MHC II, CD86 and CD40 molecules that indicate the maturation of these cells. (B) Mean normalized fluorescence intensity of expression of MHC II markers, CD86 and CD40 in BMDCs treated with CD40L VLPs. Representative image from an independent experiment. MFI: Median Fluorescence Intensity.

CD40L VLP Signaling Induces Apoptosis of Tumor Cells, Modulating Genes that Control Apoptosis and Cell Proliferation.

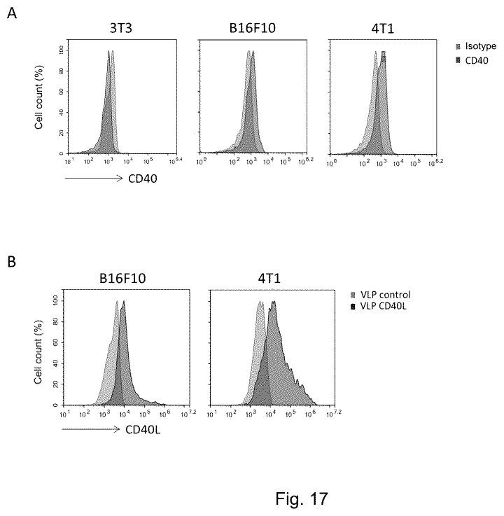

Data in the literature have already described the tumoricidal action of CD40:CD40L signaling. In this context, it was sought to demonstrate whether CD40L VLPs were capable of binding to cancer cells that overexpressed the CD40 receptor on their surface. Using a flow cytometry assay, in which CD40L VLP preparations were incubated with B16F10 and 4T1 tumor cells and stain these particles with anti-CD40L antibodies, it was found that CD40L VLPs can bind to CD40 receptors present on the cell membrane tumors, as shown in FIG. 17.

FIG. 17 shows that the CD40L VLPs have tropism for tumor cells. (A) Tumor cells overexpress the CD40 receptor. Representative histogram of flow cytometry assay of 3T3, B16F10 and 4T1 cells for CD40 receptor expression. The expression of CD40 on tumor cells is significantly higher compared to murine fibroblast cells (3T3) used as a negative control. Gray peak: cells stained with Rat IgG2a isotype antibody; Red peak: cells stained with anti-CD40 antibody. (B) CD40L VLPs bind to B16F10 and 4T1 tumor cells via CD40 receptors. CD40L expression in B16F10 and 4T1 cells is pronounced under conditions where tumor cells were incubated with CD40L VLPs and labeled with anti-CD40L antibody (blue peak). B16F10 and 4T1 cells incubated with control VLPs (gray peak) and anti-CD40L antibody showed low expression of CD40L. Representative images from two independent experiments.

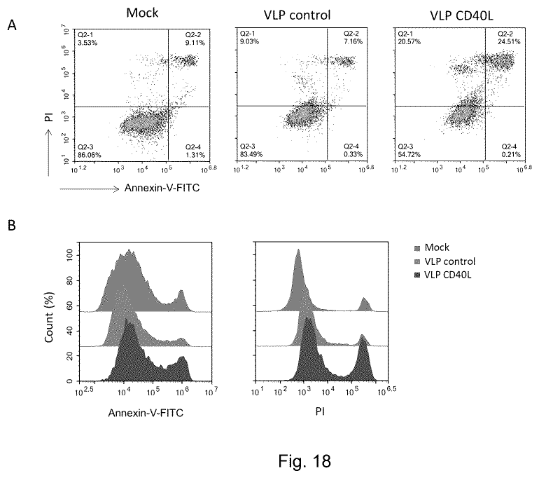

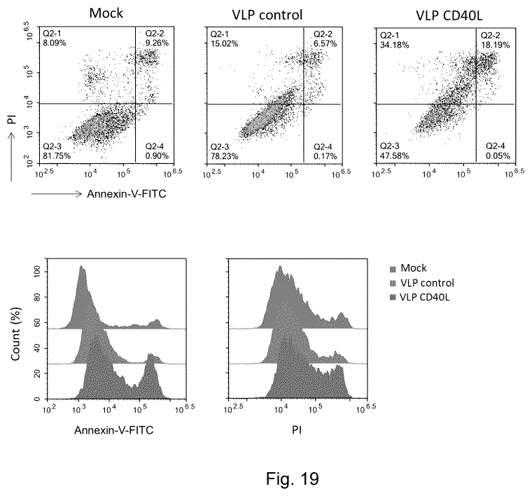

Following, it was assessed whether CD40:CD40L signaling mediated by engineered VLPs was functional and capable of inducing cancer cell apoptosis. It was observed, through an annexin-V/PI double labeling assay, that CD40L VLPs induced apoptosis of B16F10 and 4T1 tumor cells after 48 hours of incubation. In contrast, tumor cells exposed to control VLPs showed a lower percentage of apoptosis, as shown in FIGS. 18 and 19.

FIG. 18 shows that the CD40L VLPs induce apoptosis in B16F10 tumor cells. (A) Representative image of the annexin-V/PI double labeling assay. The percentage of cells undergoing apoptosis, characterized by increased annexin-V and PI fluorescence intensity, was higher under conditions in which B16F10 cells were incubated with CD40L VLPs. (B) Representative histograms of the mean fluorescence intensity (MFI) of annexin-V and PI. Representative data from an independent experiment.

FIG. 19 shows that the CD40L VLPs induce apoptosis in 4T1 tumor cells. (A) Representative image of the annexin-V/PI double labeling assay. The percentage of cells undergoing apoptosis, characterized by increased annexin-V and PI fluorescence intensity, was higher under conditions in which 4T1 cells were incubated with CD40L VLPs. (B) Representative histograms of the mean fluorescence intensity (MFI) of annexin-V and PI. Representative data from an independent experiment.

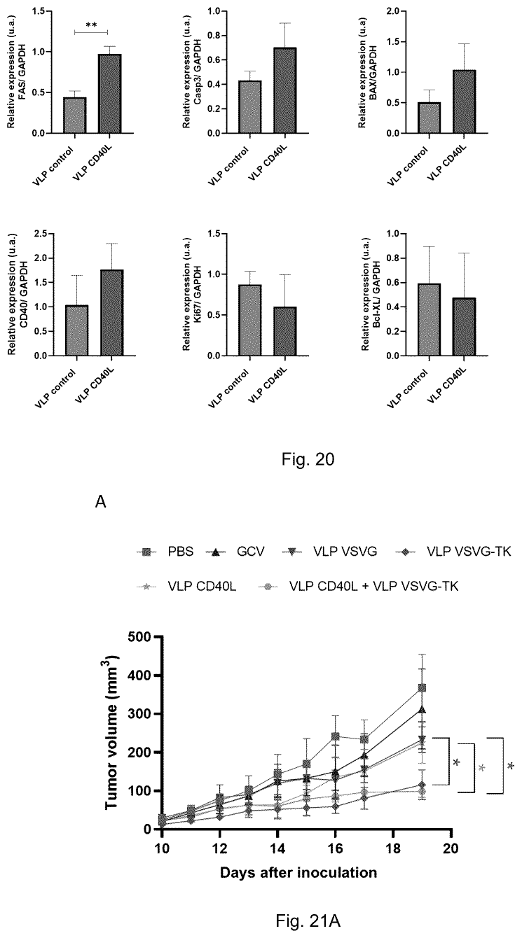

Observing that VLPs containing the CD40 ligand stimulated direct apoptosis of tumor cells, it was investigated whether CD40:CD40L signaling orchestrated by VLPs was efficient in modulating genes associated with apoptosis and cell proliferation. To do this, an RT-qPCR assay was performed, using mRNA extracted from 4T1 tumor cells that had been treated with CD40L VLPs. The results suggested that the interaction of CD40L VLPs with cancer cells can increase the expression of anti-apoptotic genes, such as FAS, BAX and Casp3, and decrease the expression of Bcl-XL and Ki67 which are associated with apoptosis inhibition and induction cell proliferation, respectively. The expression of the CD40 receptor was also higher in cells treated with CD40L VLPs, compared to cells treated with control VLPs, as shown in FIG. 20.

FIG. 20 shows that the CD40L VLPs modulate the expression of genes that induce apoptosis and inhibit the expression of genes that stimulate cell proliferation. RT-qPCR of RNA extracted from tumor cells treated with the indicated VLPs. The results represent the normalized quantitative ratio (RQ) of the expression of the genes of interest in relation to the endogenous control GAPDH. Bars indicate standard deviation from the mean. RQ=2−Δ(ΔCt). Orange bar: cells treated with control VLPs. Blue bar: cells treated with CD40L VLPs. Representative image from two independent experiments. The one-way ANOVA test, followed by the Tukey test, was used for statistical analysis, considering *p<0.05 values.

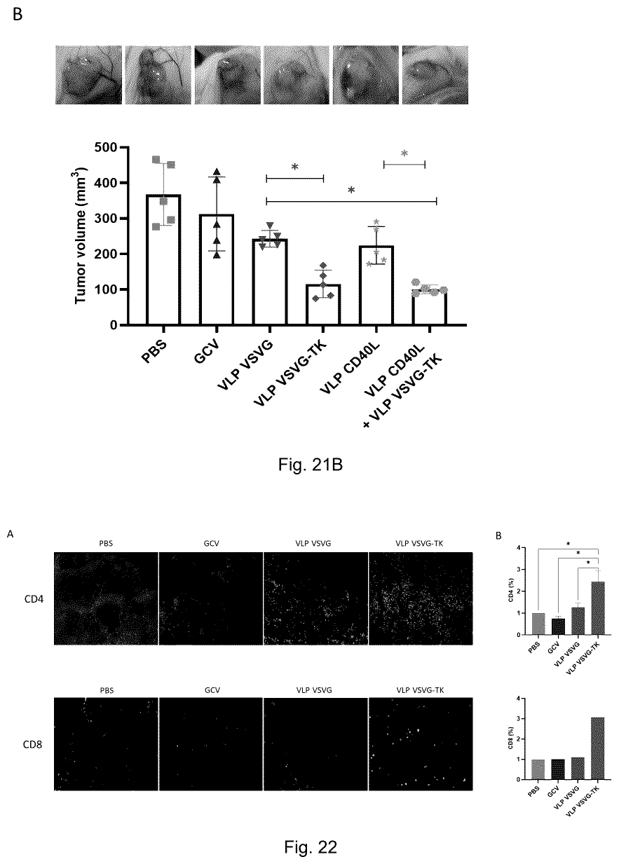

Intratumoral application of VLPs engineered with the HSV-TK enzyme and VLPs decorated with the immunomodulator CD40L reduces tumor volume in immunocompetent animals. The results obtained from in vitro assays demonstrate that VSVG-TK VLPs can act as efficient carriers of the HSV-TK suicide enzyme into tumor cells. It was found that the treatment of cancer cells in association with the prodrug ganciclovir decreased the viability and induced apoptosis of these cells. It was also observed that the supernatant from tumor cells killed by the HSV-TK/GCV system, mediated by the action of VSVG-TK VLPs, led to the activation of bone marrow-derived dendritic cells. The immunomodulatory and apoptotic function of CD40L VLPs were analyzed. The results showed that these particles can mature BMDCs and stimulate cancer cell death in vitro. In this scenario, in vivo experiments were carried out in order to observe whether preparations of VLPs VSVG-TK and CD40L, applied intratumorally, were capable of reducing tumor volume in immunocompetent animals. First, 4T1 cells were introduced subcutaneously into 36 animals. After the tumors reached an approximate size of 20 mm3, the animals were randomly separated into 6 different groups: PBS, GCV, VLP VSVG, VLP VSVG-TK, VLP CD40L and VLP VSVG-TK associated with VLP CD40L, and the treatment protocol with the different preparations of VLPs and the prodrug ganciclovir, described in detail previously, was started. At the end of the experiment, it was observed a significant reduction in tumor volume in animals that received intratumoral injections of VSVG-TK VLPs and in those that were treated with the combination of VLPs, as shown in FIGS. 21A and 21B. It was also observed that tumors treated with CD40L VLPs together with VSVG-TK VLPs showed slowed growth compared to the condition in which the animals received only VSVG-TK VLPs, although this difference was not statistically significant.

FIGS. 21A and 21B show that the treatment with VSVG-TK and CD40L VLPs decreases tumor growth. (A) 19-day tumor volume with different treatments. 1×10⁵ 4T1 triple negative breast tumor cells were injected subcutaneously into the left flank of BalB/c females. Tumor growth was observed for 19 days. After reaching an approximate size of 20 mm3, three doses of VLPs VSVG-TK, VLP CD40L and the combination of both preparations, at concentrations 1×10¹⁰, 2×10¹⁰ and 1×10¹⁰+2×10¹⁰, respectively, were injected intratumorally within seven days. The animals also received intraperitoneal injections of ganciclovir (50 mg/kg) for eight consecutive days, starting on the first day of intratumoral injection. Tumor volume was calculated by multiplying the measurements of the three dimensions (vertical, horizontal and diagonal) by the value of π and dividing the final product by 6. Two-way ANOVA test, followed by Tukey's multi-comparison test, was used for statistical analysis, considering the values of *p<0.05.

Tumors treated with VSVG-TK VLPs showed an increase in the infiltrate of CD4+ and CD8+ T cells. The next objective was to investigate whether tumors from animals treated with VSVG-TK VLPs showed an increase in the infiltrate of CD4+ and CD8+ T cells. To achieve this, immunofluorescence protocols were performed on cryosections of tumor tissues. The present findings showed a significant increase in CD4 expression in the tumors of animals treated with VSVG-TK VLPs compared to those treated with control VLPs. A higher percentage of CD8 was also observed in tumors that received intratumoral injections of VSVG-TK VLPs, as shown in FIG. 22.

FIG. 22 shows that the Local administration of VSVG-TK VLPs induced an increase in the intratumoral amount of CD4 and CD8. (A) Immunofluorescence images of cryosections of tumors from animals treated with the different VLP preparations. revealed increased percentage of CD4 and CD8 in tumors treated with VSVG-TK VLPs compared to PBS, GCV and VSVG VLP controls. (B). Percentage of CD4 and CD8 resulting from different intratumoral treatments. Representative image of three independent experiments for analyzing CD4 expression, and one independent experiment for evaluating the percentage of CD8. The data correspond to the normalized means after applying the statistical analysis using one-way ANOVA, followed by Turkey's multi-comparison test. *p values<0.05.

The spleen of animals treated with the combination of CD40L and VSVG-TK VLPs have an increased number of CD4+ T and CD8+ T lymphocytes.

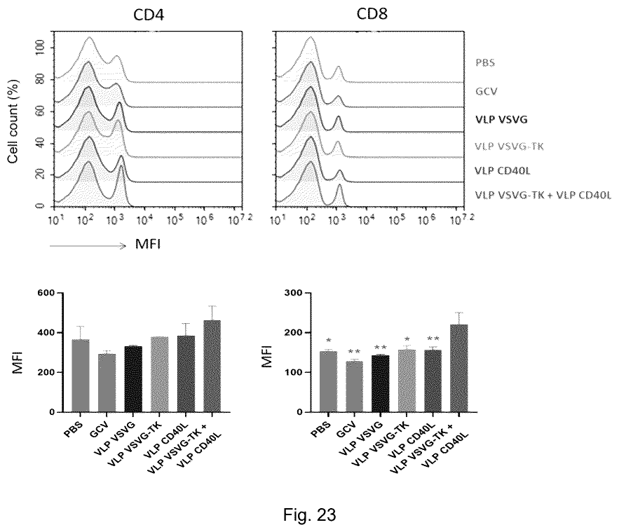

After analyzing the increase in CD4+ and CD8+ T cell infiltration in tumors from animals treated with VSVG-TK VLPs, the lymphocyte profile was investigated in the spleen of these animals. It was observed that the spleen of animals treated with VSVG-TK VLPs combined with CD40L VLPs showed a significant increase in the number of CD8+ T cells compared to animals treated only with VSVG VLPs and CD40L VLPs, as shown in FIG. 23. The increase in CD4+ T lymphocytes was also evident in animals that received intratumoral injections of VSVG-TK and CD40L VLPs, although this increase was not statistically significant.

FIG. 23 shows that the spleen of animals treated with the combination of VSVG-TK VLPs and CD40L VLPs had an increased number of CD4+T and CD8+ T lymphocytes. Flow cytometry of macerated spleens extracted from animals subjected to different intratumoral treatments. The splenocytes were marked with anti-CD4 and anti-CD8 antibodies in order to evaluate the percentage of these cells after treating the animals with the different VLP preparations. The increase in the number of CD8+ T cells was evident in animals treated with the VLPs VSVG-TK and VLP CD40L together, compared to control animals and those treated with a single type of VLP. Although the presence of CD4+ T cells was greater in animals treated with the combination, this increase was not statistically significant. MFI: mean fluorescence intensity. Representative histogram of 4 independent experiments. The data correspond to the normalized means after applying the statistical analysis using one-way ANOVA, followed by Turkey's multi-comparison test. Values of *p<0.05 and **p<0.001.

### CONCLUSION

The results presented in this work allow us to conclude that VLPs derived from lentiviral capsids can be explored as delivery systems for the suicide enzyme HSV-TK and the immunomodulatory protein CD40L. The present VLPs were characterized in biophysical assays, in vitro assays and it was also performed a proof of concept in vivo, challenging immunocompetent animals with syngeneic tumors, which were treated with therapeutic VLPs. In addition to observing a reduction in tumors in treated animals, it was also found that the combination of VLPs VSVG-TK and CD40L had an additive effect on the systemic antitumor immune response in animals, increasing the number of CD4+ and CD8+ T cells in the spleens of these animals. In this sense, other aspects of the immune response should be explored, such as the reduction of regulatory T cells and M1 and M2 macrophage markers. Additionally, in preliminary data, it was also found that the use of therapeutic VLPs could reduce tumor neovascularization, which could also be explored.

An interesting observation is that in contrast to other in vivo tumor models commonly used, in which immunomodulatory VLPs were tested being administered just 1 day after the injection of tumor cells, in tests described in this application the therapy started only after obtaining a palpable tumor, in accordance with previous methodologies (MANRIQUE-RINCON et al., 2017; PALAMETA et al., 2022). This is highly relevant to the clinical potential, in which it is necessary to eliminate tumors that are already established and in more advanced stages.

What is claimed is:

1. A composition comprising virus like particles (VLPs), wherein the composition combines two virus like particles (VLPs) capable of promoting an abscopal effect.

2. The composition of claim 1, wherein the composition comprises a VLP-derived from a viral capsid decorated with VSV-g envelope glycoprotein and loaded with Thymidine Kinase (HSV-TK) from viral herpes.

3. The composition of claim 1, wherein the composition comprises a VLP-derived from a viral capsid decorated with CD40L.

4. The composition of claim 1, wherein the composition comprises a VLP-derived from a viral capsid decorated with VSV-g envelope glycoprotein and loaded with Thymidine Kinase (HSV-TK) from viral herpes and a VLP-derived from viral capsid decorated with CD40L.

5. The composition of claim 1, wherein the VLPs are derived from a lentivirus capsid.

6. A kit for antitumoral therapy comprising:
(a) a VLP-derived from a viral capsid decorated with VSV-g envelope glycoprotein and loaded with Thymidine Kinase (HSV-TK) from viral herpes;
(b) a VLP-derived from a viral capsid decorated with CD40L; and
(c) an antiviral drug,
wherein the VLPs are derived from a lentivirus capsid.

7. The kit of claim 6, wherein the antiviral drug comprises ganciclovir.

8. A method for antitumoral therapy comprising administration of two virus like particles (VLPs) capable of promoting an abscopal effect, and an antiviral drug.

9. The method of claim 8, wherein the antiviral drug comprises ganciclovir.

10. The method of claim 8, wherein the VLPs comprise a VLP derived from a viral capsid decorated with a VSV-g envelope glycoprotein and loaded with Thymidine Kinase (HSV-TK) from viral herpes (VSVg-TK VLP) and a VLP derived from a viral capsid decorated with CD40L (CD40L VLPs).

11. The method of claim 10, wherein the VLPs are derived from a lentivirus capsid.

12. The method of claim 10, wherein VSVg-TK VLP is administered locally at a tumor site.

13. The method of claim 10, wherein CD40L VLPs is administered locally or systemically.
